(19) United States
(12) Patent Application Publication
(10) Pub. No.: US 2026/0013818 A1
(43) Pub. Date: Jan. 15, 2026

(54) SYSTEM AND METHOD OF DETERMINING OPTIMAL 3-DIMENSIONAL POSITION AND ORIENTATION OF IMAGING DEVICE FOR IMAGING PATIENT BONES

(71) Applicant: GLOBUS MEDICAL, INC., AUDUBON, PA (US)

(72) Inventor: Norbert Johnson, North Andover, MA (US)

(21) Appl. No.: 19/330,218

(22) Filed: Sep. 16, 2025

Related U.S. Application Data
(63) Continuation of application No. 17/088,853, filed on Nov. 4, 2020, now Pat. No. 12,414,752.
(60) Provisional application No. 62/977,435, filed on Feb. 17, 2020.
(60) Provisional application No. 62/990,064, filed on Mar. 16, 2020.

(51) Int. Cl. A61B 6/00; A61B 6/40; A61B 6/46; A61B 6/50; G06T 7/11; G06T 7/246; G06T 7/30; G06T 7/70; G06T 17/00; G06T 19/20

(52) U.S. Cl. CPC A61B 6/5235; A61B 6/4028; A61B 6/4441; A61B 6/466; A61B 6/505; G06T 7/11; G06T 7/246; G06T 7/30; G06T 7/70; G06T 17/00; G06T 19/20; G06T 2207/10064; G06T 2207/10081; G06T 2207/10116; G06T 2207/20221; G06T 2207/30012; G06T 2207/30204; G06T 2219/2004; G06T 2219/2016

(57) ABSTRACT

A method of determining the imaging arm's optimal 3-dimensional position and orientation for taking images of a body implant or body structure such as vertebral body is provided. Test images of vertebral body of interest are initially taken by the user and are received by the imaging device. The test images typically include AP and lateral x-ray images of the vertebral body. From the test images, the vertebral body is segmented. A 3-dimensional model of the vertebral body is then aligned against the corresponding vertebral body in the test images. Based on the alignment, a 3-dimensional position and orientation of the imaging arm for taking optimal A-P and lateral x-ray images are determined based on the aligned 3-dimensional model. The present method eliminates the need to repeatedly take fluoro shots manually to find the optimum images to thereby reduce procedural time, x-ray exposure and procedure costs.

### CROSS REFERENCE TO RELATED APPLICATIONS

This application is a continuation of U.S. patent application Ser. No. 17/088,853, filed Nov. 4, 2020, which claims priority to U.S. Provisional Application No. 62/977,435, filed Feb. 17, 2020 and U.S. Provisional Application No. 62/990,064, filed Mar. 16, 2020, all of which are incorporated herein by reference in their entirety for all purposes.

### TECHNICAL FIELD

The present disclosure relates to surgical imaging systems, and in particular, system for determining 3-dimensional position and orientation of a 2-dimensional imaging device for a robot assisted surgery.

### BACKGROUND

Surgeons use imaging of the spine to assist with accurate placement of implants such as interbody spacers and pedicle screws. Spine procedures can be open surgery where the surgeon has good visibility of the spine due to a larger incision or minimally invasive surgery (MIS) where the surgeon has no visibility of the spine due to very small incisions. As a spine procedure moves toward robot assisted or at least navigation assisted MIS, more x-ray images are needed to accurately place spinal implants. Today, C-arms are the main surgical imaging systems in use which produce 2-dimensional (2D) x-ray images (alternatively and interchangeably called “fluoro” images). A typical fluoro image is an Anterior-Posterior (AP) image (see 1902 in FIG. 19A). Given that depth cannot be determined by one 2D image, a second image which is 90-degrees offset from the AP image is taken; this is called a lateral image 1904 as seen in FIG. 19B.

It is important to align the C-arm properly with the spinal vertebral body so the surgeon can align the implant with the anatomy. FIG. 20 illustrates an oblique improperly aligned lateral image of the lumbar spine. With such a misaligned image, it would be very difficult to accurately place an implant even by an experienced surgeon.

To take optimal images, a radiological technician (RT) iteratively positions the C-arm or patient bed for each of the AP image and lateral image. After each position adjustment, a fluoro image is taken and examined. For an experienced RT, the number of shots to acquire a good AP or lateral image may be three. For an inexperienced RT, the number is greater than 10. Since each vertebral level requires two images (AP and lateral), a spine case involving three vertebral levels would need at least six fluoro shots for each level (three shots for AP and three shots for lateral) by an experienced RT, resulting in a total number of fluoro shots at 18. For an inexperienced RT, the total number of fluoro shots for the same three vertebral procedure may be higher than 60 (10 shots for AP and 10 shots for lateral).

As can be appreciated, a typical spine procedure exposes the patient, RT and surgeon to a large amount of radiation. Moreover, the large number of fluoro shots during surgery slows down the surgical procedure and creates substantially higher risk to the patients as well as higher costs.

Therefore, it would be desirable to provide a system and method for reducing the number of fluoro shots required for a spine surgery.

### SUMMARY OF THE INVENTION

To meet this and other needs, devices, systems, and methods for determining the optimal 3-dimensional position and orientation of an imaging arm of an imaging device is provided.

According to one aspect of the present invention, a method of determining the imaging arm's optimal 3-dimensional position and orientation for taking images of a vertebral body. According to the method, test images of the vertebral body are taken by the user and are received by the imaging device. The test images include an x-ray image of the vertebral body and a second x-ray image at a different angle than the first x-ray image. Typically, the two images would be taken approximately 90 degrees from each other. From the test images, vertebral bodies of interest are identified either by the user or a computer. The vertebral bodies are then segmented by the computer with or without help from the user. A 3-dimensional model of the vertebral body is then aligned against the corresponding vertebral body in the test images. Based on the alignment, a 3-dimensional position and orientation of the imaging arm for taking optimal AP and lateral x-ray images are determined based on the aligned 3-dimensional model.

By having the computer determine the optimal position and orientation of the imaging arm of the imaging device, the present method eliminates the need to repeatedly take fluoro shots manually to find the optimum images.

### BRIEF DESCRIPTION OF THE DRAWINGS

FIG. 1 is an overhead view of a potential arrangement for locations of the robotic system, patient, surgeon, and other medical personnel during a surgical procedure;

FIG. 2 illustrates the robotic system including positioning of the surgical robot and the camera relative to the patient according to one embodiment;

FIG. 3 illustrates a surgical robotic system in accordance with an exemplary embodiment;

FIG. 4 illustrates a portion of a surgical robot in accordance with an exemplary embodiment;

FIG. 5 illustrates a block diagram of a surgical robot in accordance with an exemplary embodiment;

FIG. 6 illustrates a surgical robot in accordance with an exemplary embodiment;

FIGS. 7A-7C illustrate an end-effector in accordance with an exemplary embodiment;

FIG. 8 illustrates a surgical instrument and the end-effector, before and after, inserting the surgical instrument into the guide tube of the end-effector according to one embodiment;

FIGS. 9A-9C illustrate portions of an end-effector and robot arm in accordance with an exemplary embodiment;

FIG. 10 illustrates a dynamic reference array, an imaging array, and other components in accordance with an exemplary embodiment;

FIG. 11 illustrates a method of registration in accordance with an exemplary embodiment;

FIG. 12A-12B illustrate embodiments of imaging devices according to exemplary embodiments;

FIG. 13A illustrates a portion of a robot including the robot arm and an end-effector in accordance with an exemplary embodiment;

FIG. 13B is a close-up view of the end-effector, with a plurality of tracking markers rigidly affixed thereon, shown in FIG. 13A;

FIG. 13C is a tool or instrument with a plurality of tracking markers rigidly affixed thereon according to one embodiment;

FIG. 14A is an alternative version of an end-effector with moveable tracking markers in a first configuration;

FIG. 14B is the end-effector shown in FIG. 14A with the moveable tracking markers in a second configuration;

FIG. 14C shows the template of tracking markers in the first configuration from FIG. 14A;

FIG. 14D shows the template of tracking markers in the second configuration from FIG. 14B;

FIG. 15A shows an alternative version of the end-effector having only a single tracking marker affixed thereto;

FIG. 15B shows the end-effector of FIG. 15A with an instrument disposed through the guide tube;

FIG. 15C shows the end-effector of FIG. 15A with the instrument in two different positions, and the resulting logic to determine if the instrument is positioned within the guide tube or outside of the guide tube;

FIG. 15D shows the end-effector of FIG. 15A with the instrument in the guide tube at two different frames and its relative distance to the single tracking marker on the guide tube;

FIG. 15E shows the end-effector of FIG. 15A relative to a coordinate system;

FIG. 16 is a block diagram of a method for navigating and moving the end-effector of the robot to a desired target trajectory;

FIGS. 17A-17B depict an instrument for inserting an expandable implant having fixed and moveable tracking markers in contracted and expanded positions, respectively;

FIGS. 18A-18B depict an instrument for inserting an articulating implant having fixed and moveable tracking markers in insertion and angled positions, respectively;

FIGS. 19A and 19B illustrate typical AP and lateral images of a spine;

FIG. 20 illustrates a misaligned lateral image of the spine;

FIG. 21 illustrates a method of determining the 3-dimensional position of an imaging arm of an imaging device for taking optimal images of a vertebral body according to one aspect of the present invention;

FIG. 22 illustrates a calibration ring having tracking markers according to one aspect of the present invention;

FIG. 23 is an example of an x-ray imaging device having an automatic positioning capability with respect to the 3D position and orientation of its C-arm according one aspect of the present invention;

FIG. 24A is an example of a graphical user interface showing the available selection of images at different vertebral levels according to an aspect of the present invention;

FIG. 24B is an example of a graphical user interface showing the additional available selection of images at adjacent vertebral levels according to an aspect of the present invention;

FIG. 25 is an example of a graphical user interface that guides a user to position the C-arm at an optimal position according to an aspect of the present invention;

FIG. 26 is a representation of a method of performing a segmentation of vertebral bodies according to an aspect of the present invention; and

FIGS. 27A and 27B graphically illustrate a method of aligning a 3D model of a selected vertebral body to the scanned AP and lateral images according to an aspect of the present invention.

### DETAILED DESCRIPTION OF THE INVENTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

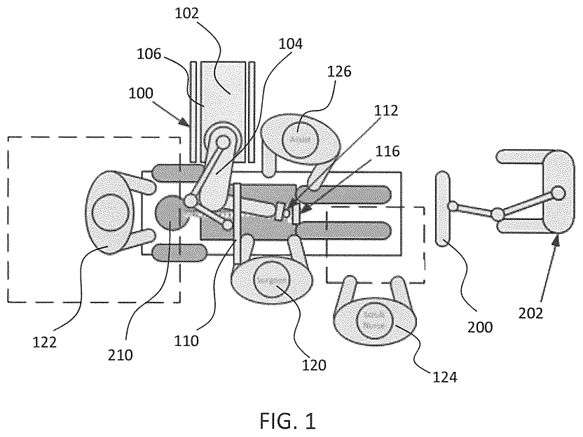

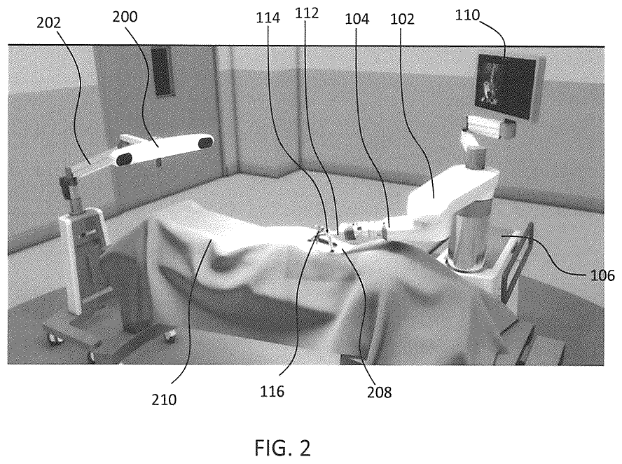

Turning now to the drawing, FIGS. 1 and 2 illustrate a surgical robot system 100 in accordance with an exemplary embodiment. Surgical robot system 100 may include, for example, a surgical robot 102, one or more robot arms 104, a base 106, a display 110, an end-effector 112, for example, including a guide tube 114, and one or more tracking markers 118. The surgical robot system 100 may include a patient tracking device 116 also including one or more tracking markers 118, which is adapted to be secured directly to the patient 210 (e.g., to the bone of the patient 210). The surgical robot system 100 may also utilize a camera 200, for example, positioned on a camera stand 202. The camera stand 202 can have any suitable configuration to move, orient, and support the camera 200 in a desired position. The camera 200 may include any suitable camera or cameras, such as one or more infrared cameras (e.g., bifocal or stereophotogrammetric cameras), able to identify, for example, active and passive tracking markers 118 in a given measurement volume viewable from the perspective of the camera 200. The camera 200 may scan the given measurement volume and detect the light that comes from the markers 118 in order to identify and determine the position of the markers 118 in three-dimensions. For example, active markers 118 may include infrared-emitting markers that are activated by an electrical signal (e.g., infrared light emitting diodes (LEDs)), and passive markers 118 may include retro-reflective markers that reflect infrared light (e.g., they reflect incoming IR radiation into the direction of the incoming light), for example, emitted by illuminators on the camera 200 or other suitable device.

FIGS. 1 and 2 illustrate a potential configuration for the placement of the surgical robot system 100 in an operating room environment. For example, the robot 102 may be positioned near or next to patient 210. Although depicted near the head of the patient 210, it will be appreciated that the robot 102 can be positioned at any suitable location near the patient 210 depending on the area of the patient 210 undergoing the operation. The camera 200 may be separated from the robot system 100 and positioned at the foot of patient 210. This location allows the camera 200 to have a direct visual line of sight to the surgical field 208. Again, it is contemplated that the camera 200 may be located at any suitable position having line of sight to the surgical field 208. In the configuration shown, the surgeon 120 may be positioned across from the robot 102, but is still able to manipulate the end-effector 112 and the display 110. A surgical assistant 126 may be positioned across from the surgeon 120 again with access to both the end-effector 112 and the display 110. If desired, the locations of the surgeon 120 and the assistant 126 may be reversed. The traditional areas for the anesthesiologist 122 and the nurse or scrub tech 124 remain unimpeded by the locations of the robot 102 and camera 200.

With respect to the other components of the robot 102, the display 110 can be attached to the surgical robot 102 and in other exemplary embodiments, display 110 can be detached from surgical robot 102, either within a surgical room with the surgical robot 102, or in a remote location. End-effector 112 may be coupled to the robot arm 104 and controlled by at least one motor. In exemplary embodiments, end-effector 112 can comprise a guide tube 114, which is able to receive and orient a surgical instrument 608 (described further herein) used to perform surgery on the patient 210. As used herein, the term “end-effector” is used interchangeably with the terms “end-effectuator” and “effectuator element.” Although generally shown with a guide tube 114, it will be appreciated that the end-effector 112 may be replaced with any suitable instrumentation suitable for use in surgery. In some embodiments, end-effector 112 can comprise any known structure for effecting the movement of the surgical instrument 608 in a desired manner.

The surgical robot 102 is able to control the translation and orientation of the end-effector 112. The robot 102 is able to move end-effector 112 along x-, y-, and z-axes, for example. The end-effector 112 can be configured for selective rotation about one or more of the x-, y-, and z-axis, and a Z Frame axis (such that one or more of the Euler Angles (e.g., roll, pitch, and/or yaw) associated with end-effector 112 can be selectively controlled). In some exemplary embodiments, selective control of the translation and orientation of end-effector 112 can permit performance of medical procedures with significantly improved accuracy compared to conventional robots that utilize, for example, a six degree of freedom robot arm comprising only rotational axes. For example, the surgical robot system 100 may be used to operate on patient 210, and robot arm 104 can be positioned above the body of patient 210, with end-effector 112 selectively angled relative to the z-axis toward the body of patient 210.

In some exemplary embodiments, the position of the surgical instrument 608 can be dynamically updated so that surgical robot 102 can be aware of the location of the surgical instrument 608 at all times during the procedure. Consequently, in some exemplary embodiments, surgical robot 102 can move the surgical instrument 608 to the desired position quickly without any further assistance from a physician (unless the physician so desires). In some further embodiments, surgical robot 102 can be configured to correct the path of the surgical instrument 608 if the surgical instrument 608 strays from the selected, preplanned trajectory. In some exemplary embodiments, surgical robot 102 can be configured to permit stoppage, modification, and/or manual control of the movement of end-effector 112 and/or the surgical instrument 608. Thus, in use, in exemplary embodiments, a physician or other user can operate the system 100, and has the option to stop, modify, or manually control the autonomous movement of end-effector 112 and/or the surgical instrument 608. Further details of surgical robot system 100 including the control and movement of a surgical instrument 608 by surgical robot 102 can be found in co-pending U.S. patent application Ser. No. 13/924,505, which is incorporated herein by reference in its entirety.

The robotic surgical system 100 can comprise one or more tracking markers 118 configured to track the movement of robot arm 104, end-effector 112, patient 210, and/or the surgical instrument 608 in three dimensions. In exemplary embodiments, a plurality of tracking markers 118 can be mounted (or otherwise secured) thereon to an outer surface of the robot 102, such as, for example and without limitation, on base 106 of robot 102, on robot arm 104, or on the end-effector 112. In exemplary embodiments, at least one tracking marker 118 of the plurality of tracking markers 118 can be mounted or otherwise secured to the end-effector 112. One or more tracking markers 118 can further be mounted (or otherwise secured) to the patient 210. In exemplary embodiments, the plurality of tracking markers 118 can be positioned on the patient 210 spaced apart from the surgical field 208 to reduce the likelihood of being obscured by the surgeon, surgical tools, or other parts of the robot 102. Further, one or more tracking markers 118 can be further mounted (or otherwise secured) to the surgical tools 608 (e.g., a screw driver, dilator, implant inserter, or the like). Thus, the tracking markers 118 enable each of the marked objects (e.g., the end-effector 112, the patient 210, and the surgical tools 608) to be tracked by the robot 102. In exemplary embodiments, system 100 can use tracking information collected from each of the marked objects to calculate the orientation and location, for example, of the end-effector 112, the surgical instrument 608 (e.g., positioned in the tube 114 of the end-effector 112), and the relative position of the patient 210.

In exemplary embodiments, one or more of markers 118 may be optical markers. In some embodiments, the positioning of one or more tracking markers 118 on end-effector 112 can maximize the accuracy of the positional measurements by serving to check or verify the position of end-effector 112. Further details of surgical robot system 100 including the control, movement and tracking of surgical robot 102 and of a surgical instrument 608 can be found in co-pending U.S. patent application Ser. No. 13/924,505, which is incorporated herein by reference in its entirety.

Exemplary embodiments include one or more markers 118 coupled to the surgical instrument 608. In exemplary embodiments, these markers 118, for example, coupled to the patient 210 and surgical instruments 608, as well as markers 118 coupled to the end-effector 112 of the robot 102 can comprise conventional infrared light-emitting diodes (LEDs) or an Optotrak® diode capable of being tracked using a commercially available infrared optical tracking system such as Optotrak®. Optotrak® is a registered trademark of Northern Digital Inc., Waterloo, Ontario, Canada. In other embodiments, markers 118 can comprise conventional reflective spheres capable of being tracked using a commercially available optical tracking system such as Polaris Spectra. Polaris Spectra is also a registered trademark of Northern Digital, Inc. In an exemplary embodiment, the markers 118 coupled to the end-effector 112 are active markers which comprise infrared light-emitting diodes which may be turned on and off, and the markers 118 coupled to the patient 210 and the surgical instruments 608 comprise passive reflective spheres.

In exemplary embodiments, light emitted from and/or reflected by markers 118 can be detected by camera 200 and can be used to monitor the location and movement of the marked objects. In alternative embodiments, markers 118 can comprise a radio-frequency and/or electromagnetic reflector or transceiver and the camera 200 can include or be replaced by a radio-frequency and/or electromagnetic transceiver.

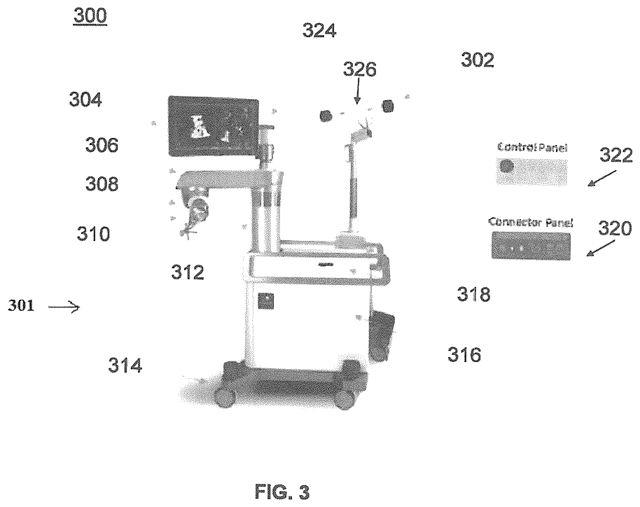

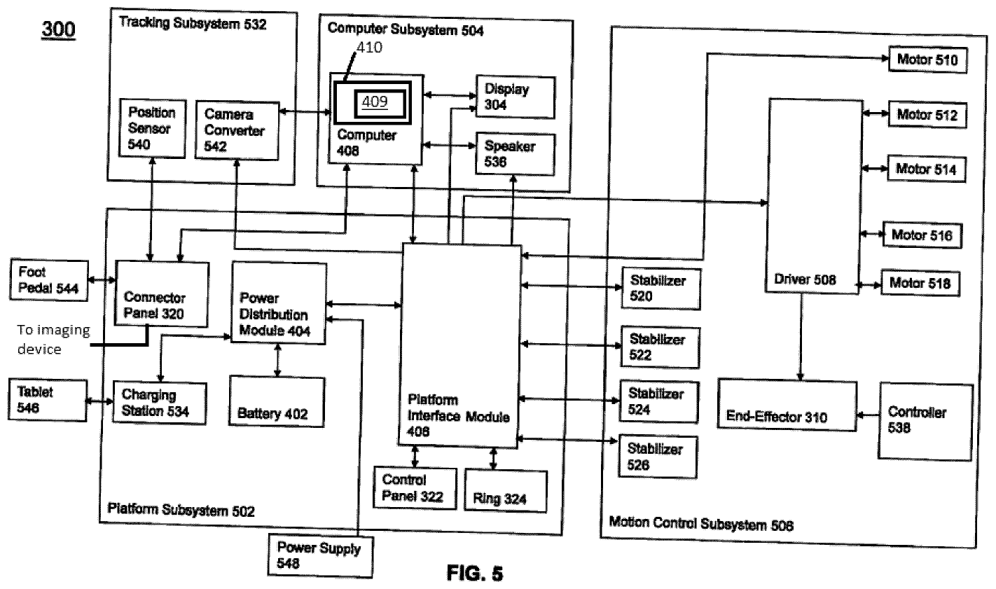

Similar to surgical robot system 100, FIG. 3 illustrates a surgical robot system 300 and camera stand 302, in a docked configuration, consistent with an exemplary embodiment of the present disclosure. Surgical robot system 300 may comprise a robot 301 including a display 304, upper arm 306, lower arm 308, end-effector 310, vertical column 312, casters 314, cabinet 316, tablet drawer 318, connector panel 320, control panel 322, and ring of information 324. Camera stand 302 may comprise camera 326. These components are described in greater with respect to FIG. 5. FIG. 3 illustrates the surgical robot system 300 in a docked configuration where the camera stand 302 is nested with the robot 301, for example, when not in use. It will be appreciated by those skilled in the art that the camera 326 and robot 301 may be separated from one another and positioned at any appropriate location during the surgical procedure, for example, as shown in FIGS. 1 and 2.

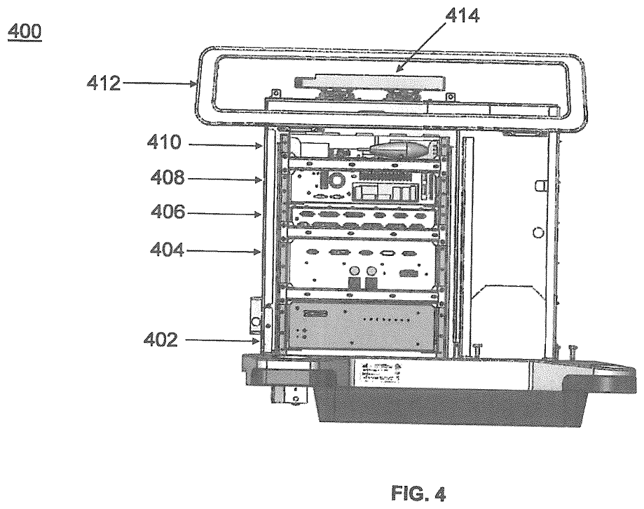

FIG. 4 illustrates a base 400 consistent with an exemplary embodiment of the present disclosure. Base 400 may be a portion of surgical robot system 300 and comprise cabinet 316. Cabinet 316 may house certain components of surgical robot system 300 including but not limited to a battery 402, a power distribution module 404, a platform interface board module 406, a computer 408, a handle 412, and a tablet drawer 414. The connections and relationship between these components is described in greater detail with respect to FIG. 5.

FIG. 5 illustrates a block diagram of certain components of an exemplary embodiment of surgical robot system 300. Surgical robot system 300 may comprise platform subsystem 502, computer subsystem 504, motion control subsystem 506, and tracking subsystem 532. Platform subsystem 502 may further comprise battery 402, power distribution module 404, platform interface board module 406, and tablet charging station 534. Computer subsystem 504 may further comprise computer 408, display 304, and speaker 536. Motion control subsystem 506 may further comprise driver circuit 508, motors 510, 512, 514, 516, 518, stabilizers 520, 522, 524, 526, end-effector 310, and controller 538. Tracking subsystem 532 may further comprise position sensor 540 and camera converter 542. System 300 may also comprise a foot pedal 544 and tablet 546.

Input power is supplied to system 300 via a power source 548 which may be provided to power distribution module 404. Power distribution module 404 receives input power and is configured to generate different power supply voltages that are provided to other modules, components, and subsystems of system 300. Power distribution module 404 may be configured to provide different voltage supplies to platform interface module 406, which may be provided to other components such as computer 408, display 304, speaker 536, driver 508 to, for example, power motors 512, 514, 516, 518 and end-effector 310, motor 510, ring 324, camera converter 542, and other components for system 300 for example, fans for cooling the electrical components within cabinet 316.

Power distribution module 404 may also provide power to other components such as tablet charging station 534 that may be located within tablet drawer 318. Tablet charging station 534 may be in wireless or wired communication with tablet 546 for charging table 546. Tablet 546 may be used by a surgeon consistent with the present disclosure and described herein.

Power distribution module 404 may also be connected to battery 402, which serves as temporary power source in the event that power distribution module 404 does not receive power from input power 548. At other times, power distribution module 404 may serve to charge battery 402 if necessary.

Other components of platform subsystem 502 may also include connector panel 320, control panel 322, and ring 324. Connector panel 320 may serve to connect different devices and components to system 300 and/or associated components and modules. Connector panel 320 may contain one or more ports that receive lines or connections from different components. For example, connector panel 320 may have a ground terminal port that may ground system 300 to other equipment, a port to connect foot pedal 544 to system 300, a port to connect to tracking subsystem 532, which may comprise position sensor 540, camera converter 542, and cameras 326 associated with camera stand 302. [A PORT IN THE CONNECTOR PANEL 320 MAY ALSO CONNECT TO AN IMAGING DEVICE FOR RECEIVING SCANNED IMAGES AND FOR CONTROLLING THE LOCATION AND ORIENTATION OF THE C-ARM BASED ON THE OPTICAL/NAVIGATION MARKERS ATTACHED TO THE IMAGING DEVICE]Connector panel 320 may also include other ports to allow USB, Ethernet, HDMI communications to other components, such as computer 408.

Control panel 322 may provide various buttons or indicators that control operation of system 300 and/or provide information regarding system 300. For example, control panel 322 may include buttons to power on or off system 300, lift or lower vertical column 312, and lift or lower stabilizers 520-526 that may be designed to engage casters 314 to lock system 300 from physically moving. Other buttons may stop system 300 in the event of an emergency, which may remove all motor power and apply mechanical brakes to stop all motion from occurring. Control panel 322 may also have indicators notifying the user of certain system conditions such as a line power indicator or status of charge for battery 402.

Ring 324 may be a visual indicator to notify the user of system 300 of different modes that system 300 is operating under and certain warnings to the user.

Computer subsystem 504 includes computer 408, display 304, and speaker 536. Computer 504 includes an operating system and software to operate system 300. Computer 504 may receive and process information from other components (for example, tracking subsystem 532, platform subsystem 502, and/or motion control subsystem 506) in order to display information to the user. Further, computer subsystem 504 may also include speaker 536 to provide audio to the user.

Tracking subsystem 532 may include position sensor 504 and converter 542. Tracking subsystem 532 may correspond to camera stand 302 including camera 326 as described with respect to FIG. 3. Position sensor 504 may be camera 326. Tracking subsystem may track the location of certain markers that are located on the different components of system 300 and/or instruments used by a user during a surgical procedure. This tracking may be conducted in a manner consistent with the present disclosure including the use of infrared technology that tracks the location of active or passive elements, such as LEDs or reflective markers, respectively. The location, orientation, and position of structures having these types of markers may be provided to computer 408 which may be shown to a user on display 304. For example, a surgical instrument 608 having these types of markers and tracked in this manner (which may be referred to as a navigational space) may be shown to a user in relation to a three dimensional image of a patient's anatomical structure.

Motion control subsystem 506 may be configured to physically move vertical column 312, upper arm 306, lower arm 308, or rotate end-effector 310. The physical movement may be conducted through the use of one or more motors 510-518. For example, motor 510 may be configured to vertically lift or lower vertical column 312. Motor 512 may be configured to laterally move upper arm 308 around a point of engagement with vertical column 312 as shown in FIG. 3. Motor 514 may be configured to laterally move lower arm 308 around a point of engagement with upper arm 308 as shown in FIG. 3. Motors 516 and 518 may be configured to move end-effector 310 in a manner such that one may control the roll and one may control the tilt, thereby providing multiple angles that end-effector 310 may be moved. These movements may be achieved by controller 538 which may control these movements through load cells disposed on end-effector 310 and activated by a user engaging these load cells to move system 300 in a desired manner.

Moreover, system 300 may provide for automatic movement of vertical column 312, upper arm 306, and lower arm 308 through a user indicating on display 304 (which may be a touchscreen input device) the location of a surgical instrument or component on three dimensional image of the patient's anatomy on display 304. The user may initiate this automatic movement by stepping on foot pedal 544 or some other input means.

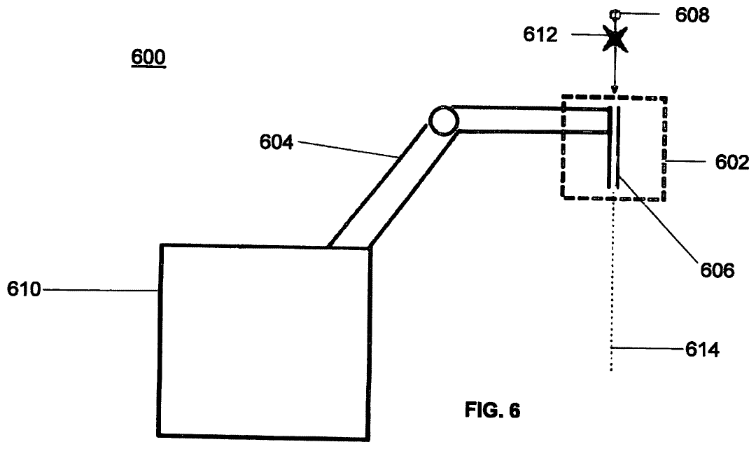

FIG. 6 illustrates a surgical robot system 600 consistent with an exemplary embodiment. Surgical robot system 600 may comprise end-effector 602, robot arm 604, guide tube 606, instrument 608, and robot base 610. Instrument tool 608 may be attached to a tracking array 612 including one or more tracking markers (such as markers 118) and have an associated trajectory 614. Trajectory 614 may represent a path of movement that instrument tool 608 is configured to travel once it is positioned through or secured in guide tube 606, for example, a path of insertion of instrument tool 608 into a patient. In an exemplary operation, robot base 610 may be configured to be in electronic communication with robot arm 604 and end-effector 602 so that surgical robot system 600 may assist a user (for example, a surgeon) in operating on the patient 210. Surgical robot system 600 may be consistent with previously described surgical robot system 100 and 300.

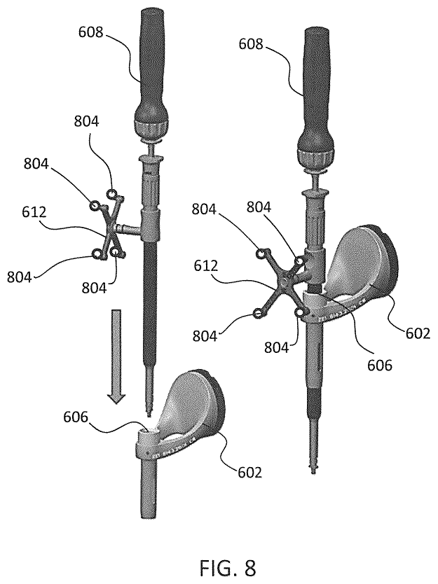

A tracking array 612 may be mounted on instrument 608 to monitor the location and orientation of instrument tool 608. The tracking array 612 may be attached to an instrument 608 and may comprise tracking markers 804. As best seen in FIG. 8, tracking markers 804 may be, for example, light emitting diodes and/or other types of reflective markers (e.g., markers 118 as described elsewhere herein). The tracking devices may be one or more line of sight devices associated with the surgical robot system. As an example, the tracking devices may be one or more cameras 200, 326 associated with the surgical robot system 100, 300 and may also track tracking array 612 for a defined domain or relative orientations of the instrument 608 in relation to the robot arm 604, the robot base 610, end-effector 602, and/or the patient 210. The tracking devices may be consistent with those structures described in connection with camera stand 302 and tracking subsystem 532.

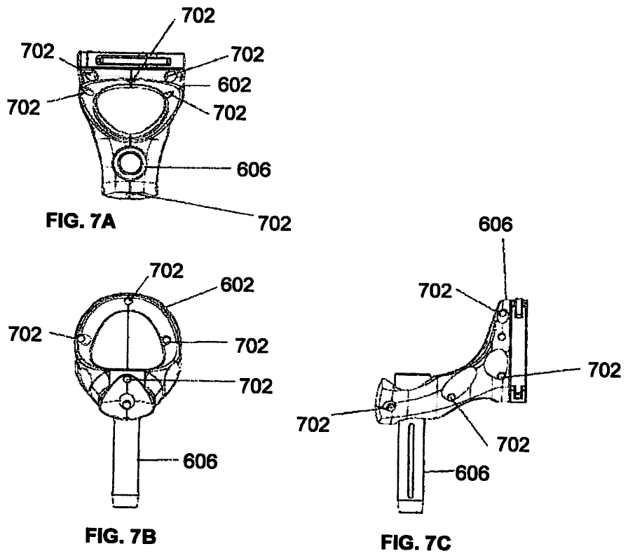

FIGS. 7A, 7B, and 7C illustrate a top view, front view, and side view, respectively, of end-effector 602 consistent with an exemplary embodiment. End-effector 602 may comprise one or more tracking markers 702. Tracking markers 702 may be light emitting diodes or other types of active and passive markers, such as tracking markers 118 that have been previously described. In an exemplary embodiment, the tracking markers 702 are active infrared-emitting markers that are activated by an electrical signal (e.g., infrared light emitting diodes (LEDs)). Thus, tracking markers 702 may be activated such that the infrared markers 702 are visible to the camera 200, 326 or may be deactivated such that the infrared markers 702 are not visible to the camera 200, 326. Thus, when the markers 702 are active, the end-effector 602 may be controlled by the system 100, 300, 600, and when the markers 702 are deactivated, the end-effector 602 may be locked in position and unable to be moved by the system 100, 300, 600.

Markers 702 may be disposed on or within end-effector 602 in a manner such that the markers 702 are visible by one or more cameras 200, 326 or other tracking devices associated with the surgical robot system 100, 300, 600. The camera 200, 326 or other tracking devices may track end-effector 602 as it moves to different positions and viewing angles by following the movement of tracking markers 702. The location of markers 702 and/or end-effector 602 may be shown on a display 110, 304 associated with the surgical robot system 100, 300, 600, for example, display 110 as shown in FIG. 2 and/or display 304 shown in FIG. 3. This display 110, 304 may allow a user to ensure that end-effector 602 is in a desirable position in relation to robot arm 604, robot base 610, the patient 210, and/or the user.

For example, as shown in FIG. 7A, markers 702 may be placed around the surface of end-effector 602 so that a tracking device placed away from the surgical field 208 and facing toward the robot 102, 301 and the camera 200, 326 is able to view at least 3 of the markers 702 through a range of common orientations of the end-effector 602 relative to the tracking device 100, 300, 600. For example, distribution of markers 702 in this way allows end-effector 602 to be monitored by the tracking devices when end-effector 602 is translated and rotated in the surgical field 208.

In addition, in exemplary embodiments, end-effector 602 may be equipped with infrared (IR) receivers that can detect when an external camera 200, 326 is getting ready to read markers 702. Upon this detection, end-effector 602 may then illuminate markers 702. The detection by the IR receivers that the external camera200, 326 is ready to read markers 702 may signal the need to synchronize a duty cycle of markers 702, which may be light emitting diodes, to an external camera200, 326. This may also allow for lower power consumption by the robotic system as a whole, whereby markers 702 would only be illuminated at the appropriate time instead of being illuminated continuously. Further, in exemplary embodiments, markers 702 may be powered off to prevent interference with other navigation tools, such as different types of surgical instruments 608.

FIG. 8 depicts one type of surgical instrument 608 including a tracking array 612 and tracking markers 804. Tracking markers 804 may be of any type described herein including but not limited to light emitting diodes or reflective spheres. Markers 804 are monitored by tracking devices associated with the surgical robot system 100, 300, 600 and may be one or more of the line of sight cameras 200, 326. The cameras 200, 326 may track the location of instrument 608 based on the position and orientation of tracking array 612 and markers 804. A user, such as a surgeon 120, may orient instrument 608 in a manner so that tracking array 612 and markers 804 are sufficiently recognized by the tracking device or camera 200, 326 to display instrument 608 and markers 804 on, for example, display 110 of the exemplary surgical robot system.

The manner in which a surgeon 120 may place instrument 608 into guide tube 606 of the end-effector 602 and adjust the instrument 608 is evident in FIG. 8. The hollow tube or guide tube 114, 606 of the end-effector 112, 310, 602 is sized and configured to receive at least a portion of the surgical instrument 608. The guide tube 114, 606 is configured to be oriented by the robot arm 104 such that insertion and trajectory for the surgical instrument 608 is able to reach a desired anatomical target within or upon the body of the patient 210. The surgical instrument 608 may include at least a portion of a generally cylindrical instrument. Although a screw driver is exemplified as the surgical tool 608, it will be appreciated that any suitable surgical tool 608 may be positioned by the end-effector 602. By way of example, the surgical instrument 608 may include one or more of a guide wire, cannula, a retractor, a drill, a reamer, a screw driver, an insertion tool, a removal tool, or the like. Although the hollow tube 114, 606 is generally shown as having a cylindrical configuration, it will be appreciated by those of skill in the art that the guide tube 114, 606 may have any suitable shape, size and configuration desired to accommodate the surgical instrument 608 and access the surgical site.

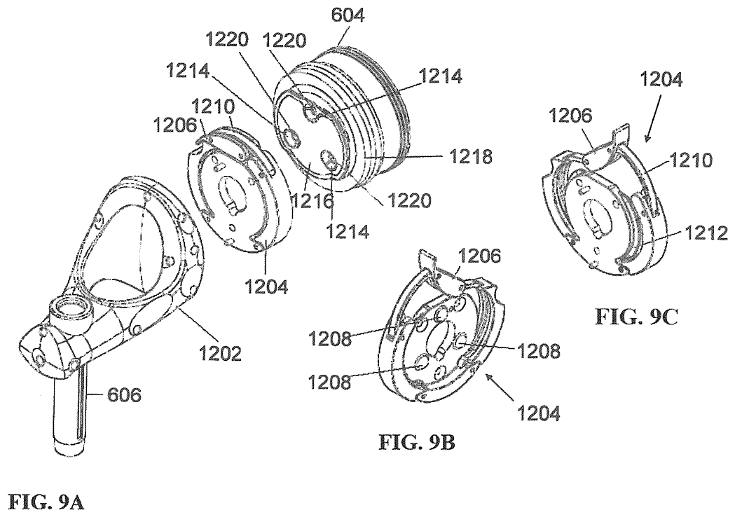

FIGS. 9A-9C illustrate end-effector 602 and a portion of robot arm 604 consistent with an exemplary embodiment. End-effector 602 may further comprise body 1202 and clamp 1204. Clamp 1204 may comprise handle 1206, balls 1208, spring 1210, and lip 1212. Robot arm 604 may further comprise depressions 1214, mounting plate 1216, lip 1218, and magnets 1220.

End-effector 602 may mechanically interface and/or engage with the surgical robot system and robot arm 604 through one or more couplings. For example, end-effector 602 may engage with robot arm 604 through a locating coupling and/or a reinforcing coupling. Through these couplings, end-effector 602 may fasten with robot arm 604 outside a flexible and sterile barrier. In an exemplary embodiment, the locating coupling may be a magnetically kinematic mount and the reinforcing coupling may be a five bar over center clamping linkage.

With respect to the locating coupling, robot arm 604 may comprise mounting plate 1216, which may be non-magnetic material, one or more depressions 1214, lip 1218, and magnets 1220. Magnet 1220 is mounted below each of depressions 1214. Portions of clamp 1204 may comprise magnetic material and be attracted by one or more magnets 1220. Through the magnetic attraction of clamp 1204 and robot arm 604, balls 1208 become seated into respective depressions 1214. For example, balls 1208 as shown in FIG. 9B would be seated in depressions 1214 as shown in FIG. 9A. This seating may be considered a magnetically-assisted kinematic coupling. Magnets 1220 may be configured to be strong enough to support the entire weight of end-effector 602 regardless of the orientation of end-effector 602. The locating coupling may be any style of kinematic mount that uniquely restrains six degrees of freedom.

With respect to the reinforcing coupling, portions of clamp 1204 may be configured to be a fixed ground link and as such clamp 1204 may serve as a five bar linkage. Closing clamp handle 1206 may fasten end-effector 602 to robot arm 604 as lip 1212 and lip 1218 engage clamp 1204 in a manner to secure end-effector 602 and robot arm 604. When clamp handle 1206 is closed, spring 1210 may be stretched or stressed while clamp 1204 is in a locked position. The locked position may be a position that provides for linkage past center. Because of a closed position that is past center, the linkage will not open absent a force applied to clamp handle 1206 to release clamp 1204. Thus, in a locked position end-effector 602 may be robustly secured to robot arm 604.

Spring 1210 may be a curved beam in tension. Spring 1210 may be comprised of a material that exhibits high stiffness and high yield strain such as virgin PEEK (poly-ether-ether-ketone). The linkage between end-effector 602 and robot arm 604 may provide for a sterile barrier between end-effector 602 and robot arm 604 without impeding fastening of the two couplings.

The reinforcing coupling may be a linkage with multiple spring members. The reinforcing coupling may latch with a cam or friction based mechanism. The reinforcing coupling may also be a sufficiently powerful electromagnet that will support fastening end-effector 102 to robot arm 604. The reinforcing coupling may be a multi-piece collar completely separate from either end-effector 602 and/or robot arm 604 that slips over an interface between end-effector 602 and robot arm 604 and tightens with a screw mechanism, an over center linkage, or a cam mechanism.

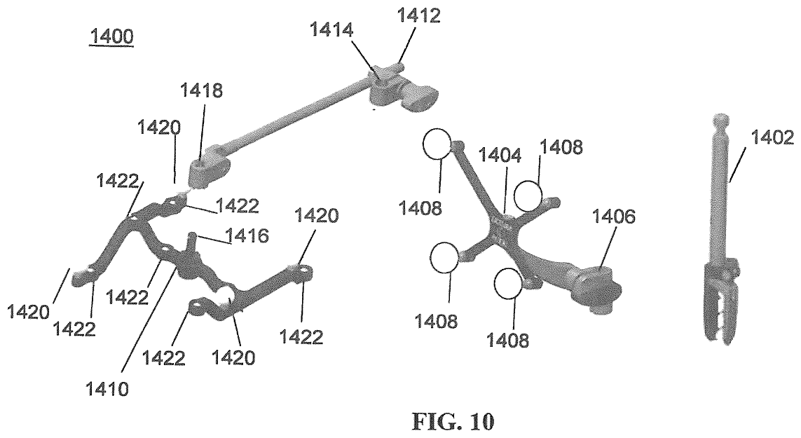

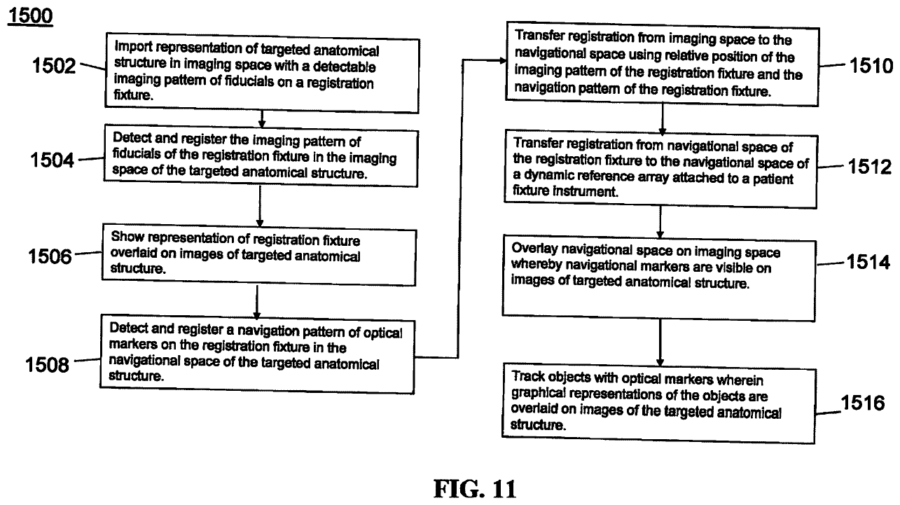

Referring to FIGS. 10 and 11, prior to or during a surgical procedure, certain registration procedures may be conducted in order to track objects and a target anatomical structure of the patient 210 both in a navigation space and an image space. In order to conduct such registration, a registration system 1400 may be used as illustrated in FIG. 10.

In order to track the position of the patient 210, a patient tracking device 116 may include a patient fixation instrument 1402 to be secured to a rigid anatomical structure of the patient 210 and a dynamic reference base (DRB) 1404 may be securely attached to the patient fixation instrument 1402. For example, patient fixation instrument 1402 may be inserted into opening 1406 of dynamic reference base 1404. Dynamic reference base 1404 may contain markers 1408 that are visible to tracking devices, such as tracking subsystem 532. These markers 1408 may be optical markers or reflective spheres, such as tracking markers 118, as previously discussed herein.

Patient fixation instrument 1402 is attached to a rigid anatomy of the patient 210 and may remain attached throughout the surgical procedure. In an exemplary embodiment, patient fixation instrument 1402 is attached to a rigid area of the patient 210, for example, a bone that is located away from the targeted anatomical structure subject to the surgical procedure. In order to track the targeted anatomical structure, dynamic reference base 1404 is associated with the targeted anatomical structure through the use of a registration fixture that is temporarily placed on or near the targeted anatomical structure in order to register the dynamic reference base 1404 with the location of the targeted anatomical structure.

A registration fixture 1410 is attached to patient fixation instrument 1402 through the use of a pivot arm 1412. Pivot arm 1412 is attached to patient fixation instrument 1402 by inserting patient fixation instrument 1402 through an opening 1414 of registration fixture 1410. Pivot arm 1412 is attached to registration fixture 1410 by, for example, inserting a knob 1416 through an opening 1418 of pivot arm 1412.

Using pivot arm 1412, registration fixture 1410 may be placed over the targeted anatomical structure and its location may be determined in an image space and navigation space using tracking markers 1420 and/or fiducials 1422 on registration fixture 1410. Registration fixture 1410 may contain a collection of markers 1420 that are visible in a navigational space (for example, markers 1420 may be detectable by tracking subsystem 532). Tracking markers 1420 may be optical markers visible in infrared light as previously described herein. Registration fixture 1410 may also contain a collection of fiducials 1422, for example, such as bearing balls, that are visible in an imaging space (for example, a three dimension CT image). As described in greater detail with respect to FIG. 11, using registration fixture 1410, the targeted anatomical structure may be associated with dynamic reference base 1404 thereby allowing depictions of objects in the navigational space to be overlaid on images of the anatomical structure. Dynamic reference base 1404, located at a position away from the targeted anatomical structure, may become a reference point thereby allowing removal of registration fixture 1410 and/or pivot arm 1412 from the surgical area.

FIG. 11 provides an exemplary method 1500 for registration consistent with the present disclosure. Method 1500 begins at step 1502 wherein a graphical representation (or image(s)) of the targeted anatomical structure may be imported into system 100, 300 600, for example computer 408. The graphical representation may be three dimensional CT or a fluoroscope scan of the targeted anatomical structure of the patient 210 which includes registration fixture 1410 and a detectable imaging pattern of fiducials 1420.

At step 1504, an imaging pattern of fiducials 1420 is detected and registered in the imaging space and stored in computer 408. Optionally, at this time at step 1506, a graphical representation of the registration fixture 1410 may be overlaid on the images of the targeted anatomical structure.

At step 1508, a navigational pattern of registration fixture 1410 is detected and registered by recognizing markers 1420. Markers 1420 may be optical markers that are recognized in the navigation space through infrared light by tracking subsystem 532 via position sensor 540. Thus, the location, orientation, and other information of the targeted anatomical structure is registered in the navigation space. Therefore, registration fixture 1410 may be recognized in both the image space through the use of fiducials 1422 and the navigation space through the use of markers 1420. At step 1510, the registration of registration fixture 1410 in the image space is transferred to the navigation space. This transferal is done, for example, by using the relative position of the imaging pattern of fiducials 1422 compared to the position of the navigation pattern of markers 1420.

At step 1512, registration of the navigation space of registration fixture 1410 (having been registered with the image space) is further transferred to the navigation space of dynamic registration array 1404 attached to patient fixture instrument 1402. Thus, registration fixture 1410 may be removed and dynamic reference base 1404 may be used to track the targeted anatomical structure in both the navigation and image space because the navigation space is associated with the image space.

At steps 1514 and 1516, the navigation space may be overlaid on the image space and objects with markers visible in the navigation space (for example, surgical instruments 608 with optical markers 804). The objects may be tracked through graphical representations of the surgical instrument 608 on the images of the targeted anatomical structure.

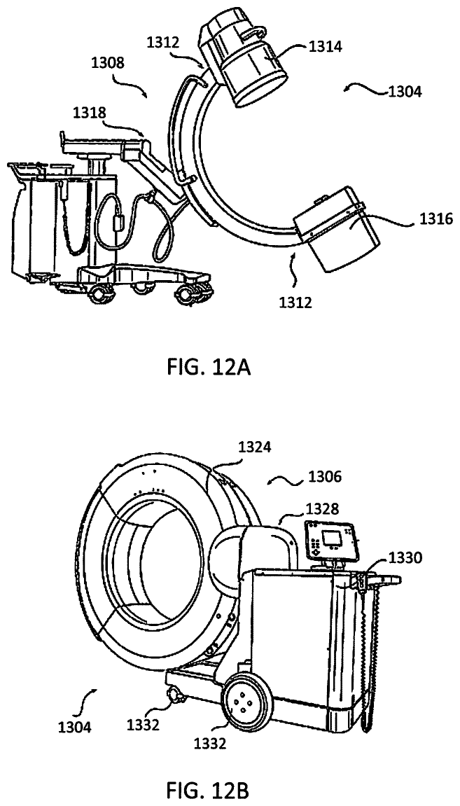

FIGS. 12A-12B illustrate imaging devices 1304 that may be used in conjunction with robot systems 100, 300, 600 to acquire pre-operative, intra-operative, post-operative, and/or real-time image data of patient 210. Any appropriate subject matter may be imaged for any appropriate procedure using the imaging system 1304. The imaging system 1304 may be any imaging device such as imaging device 1306 and/or a C-arm 1308 device. It may be desirable to take x-rays of patient 210 from a number of different positions, without the need for frequent manual repositioning of patient 210 which may be required in an x-ray system. As illustrated in FIG. 12A, the imaging system 1304 may be in the form of a C-arm 1308 that includes an elongated C-shaped member terminating in opposing distal ends 1312 of the “C” shape. C-shaped member 1130 may further comprise an x-ray source 1314 and an image receptor 1316. The space within C-arm 1308 of the arm may provide room for the physician to attend to the patient substantially free of interference from x-ray support structure 1318. As illustrated in FIG. 12B, the imaging system may include imaging device 1306 having a gantry housing 1324 attached to a support structure imaging device support structure 1328, such as a wheeled mobile cart 1330 with wheels 1332, which may enclose an image capturing portion, not illustrated. The image capturing portion may include an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of patient 210 to be acquired from multiple directions or in multiple planes. Although certain imaging systems 1304 are exemplified herein, it will be appreciated that any suitable imaging system may be selected by one of ordinary skill in the art.

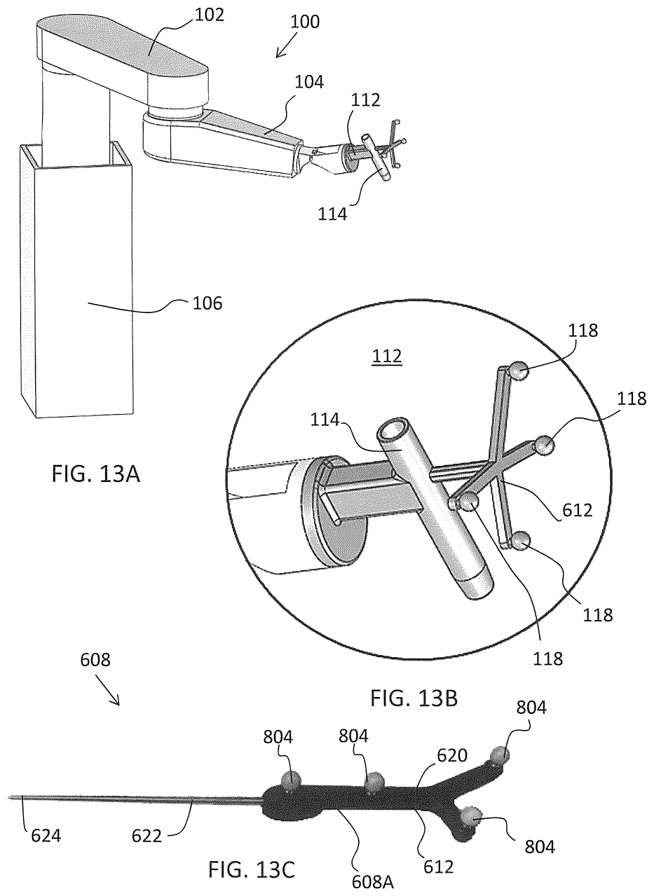

Turning now to FIGS. 13A-13C, the surgical robot system 100, 300, 600 relies on accurate positioning of the end-effector 112, 602, surgical instruments 608, and/or the patient 210 (e.g., patient tracking device 116) relative to the desired surgical area. In the embodiments shown in FIGS. 13A-13C, the tracking markers 118, 804 are rigidly attached to a portion of the instrument 608 and/or end-effector 112.

FIG. 13A depicts part of the surgical robot system 100 with the robot 102 including base 106, robot arm 104, and end-effector 112. The other elements, not illustrated, such as the display, cameras, etc. may also be present as described herein. FIG. 13B depicts a close-up view of the end-effector 112 with guide tube 114 and a plurality of tracking markers 118 rigidly affixed to the end-effector 112. In this embodiment, the plurality of tracking markers 118 are attached to the guide tube 112. FIG. 13C depicts an instrument 608 (in this case, a probe 608A) with a plurality of tracking markers 804 rigidly affixed to the instrument 608. As described elsewhere herein, the instrument 608 could include any suitable surgical instrument, such as, but not limited to, guide wire, cannula, a retractor, a drill, a reamer, a screw driver, an insertion tool, a removal tool, or the like.

When tracking an instrument 608, end-effector 112, or other object to be tracked in 3D, an array of tracking markers 118, 804 may be rigidly attached to a portion of the tool 608 or end-effector 112. Preferably, the tracking markers 118, 804 are attached such that the markers 118, 804 are out of the way (e.g., not impeding the surgical operation, visibility, etc.). The markers 118, 804 may be affixed to the instrument 608, end-effector 112, or other object to be tracked, for example, with an array 612. Usually three or four markers 118, 804 are used with an array 612. The array 612 may include a linear section, a cross piece, and may be asymmetric such that the markers 118, 804 are at different relative positions and locations with respect to one another. For example, as shown in FIG. 13C, a probe 608A with a 4-marker tracking array 612 is shown, and FIG. 13B depicts the end-effector 112 with a different 4-marker tracking array 612.

In FIG. 13C, the tracking array 612 functions as the handle 620 of the probe 608A. Thus, the four markers 804 are attached to the handle 620 of the probe 608A, which is out of the way of the shaft 622 and tip 624. Stereophotogrammetric tracking of these four markers 804 allows the instrument 608 to be tracked as a rigid body and for the tracking system 100, 300, 600 to precisely determine the position of the tip 624 and the orientation of the shaft 622 while the probe 608A is moved around in front of tracking cameras 200, 326.

To enable automatic tracking of one or more tools 608, end-effector 112, or other object to be tracked in 3D (e.g., multiple rigid bodies), the markers 118, 804 on each tool 608, end-effector 112, or the like, are arranged asymmetrically with a known inter-marker spacing. The reason for asymmetric alignment is so that it is unambiguous which marker 118, 804 corresponds to a particular location on the rigid body and whether markers 118, 804 are being viewed from the front or back, i.e., mirrored. For example, if the markers 118, 804 were arranged in a square on the tool 608 or end-effector 112, it would be unclear to the system 100, 300, 600 which marker 118, 804 corresponded to which corner of the square. For example, for the probe 608A, it would be unclear which marker 804 was closest to the shaft 622. Thus, it would be unknown which way the shaft 622 was extending from the array 612. Accordingly, each array 612 and thus each tool 608, end-effector 112, or other object to be tracked should have a unique marker pattern to allow it to be distinguished from other tools 608 or other objects being tracked. Asymmetry and unique marker patterns allow the system 100, 300, 600 to detect individual markers 118, 804 then to check the marker spacing against a stored template to determine which tool 608, end effector 112, or other object they represent. Detected markers 118, 804 can then be sorted automatically and assigned to each tracked object in the correct order. Without this information, rigid body calculations could not then be performed to extract key geometric information, for example, such as tool tip 624 and alignment of the shaft 622, unless the user manually specified which detected marker 118, 804 corresponded to which position on each rigid body. These concepts are commonly known to those skilled in the methods of 3D optical tracking.

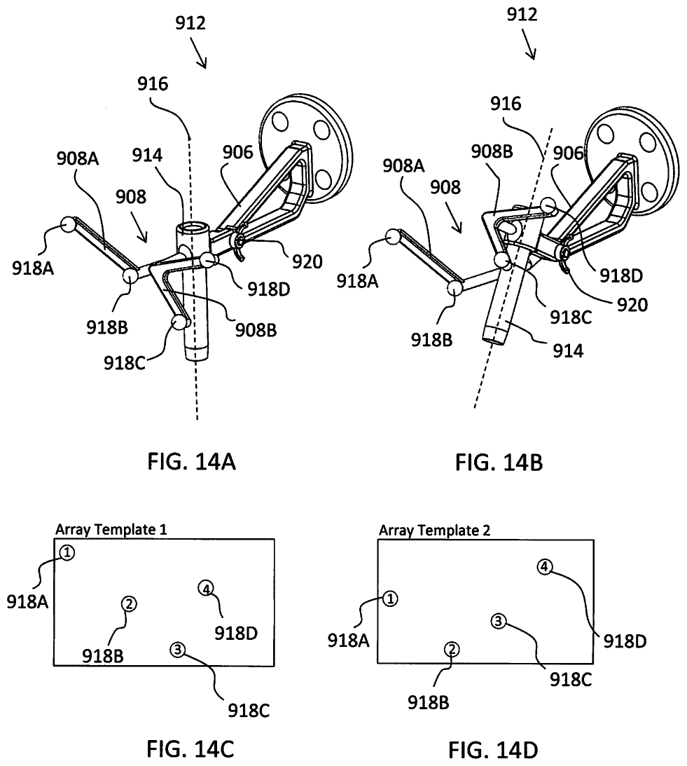

Turning now to FIGS. 14A-14D, an alternative version of an end-effector 912 with moveable tracking markers 918A-918D is shown. In FIG. 14A, an array with moveable tracking markers 918A-918D are shown in a first configuration, and in FIG. 14B the moveable tracking markers 918A-918D are shown in a second configuration, which is angled relative to the first configuration. FIG. 14C shows the template of the tracking markers 918A-918D, for example, as seen by the cameras 200, 326 in the first configuration of FIG. 14A; and FIG. 14D shows the template of tracking markers 918A-918D, for example, as seen by the cameras 200, 326 in the second configuration of FIG. 14B.

In this embodiment, 4-marker array tracking is contemplated wherein the markers 918A-918D are not all in fixed position relative to the rigid body and instead, one or more of the array markers 918A-918D can be adjusted, for example, during testing, to give updated information about the rigid body that is being tracked without disrupting the process for automatic detection and sorting of the tracked markers 918A-918D.

When tracking any tool, such as a guide tube 914 connected to the end effector 912 of a robot system 100, 300, 600, the tracking array's primary purpose is to update the position of the end effector 912 in the camera coordinate system. When using the rigid system, for example, as shown in FIG. 13B, the array 612 of reflective markers 118 rigidly extend from the guide tube 114. Because the tracking markers 118 are rigidly connected, knowledge of the marker locations in the camera coordinate system also provides exact location of the centerline, tip, and tail of the guide tube 114 in the camera coordinate system. Typically, information about the position of the end effector 112 from such an array 612 and information about the location of a target trajectory from another tracked source are used to calculate the required moves that must be input for each axis of the robot 102 that will move the guide tube 114 into alignment with the trajectory and move the tip to a particular location along the trajectory vector.

Sometimes, the desired trajectory is in an awkward or unreachable location, but if the guide tube 114 could be swiveled, it could be reached. For example, a very steep trajectory pointing away from the base 106 of the robot 102 might be reachable if the guide tube 114 could be swiveled upward beyond the limit of the pitch (wrist up-down angle) axis, but might not be reachable if the guide tube 114 is attached parallel to the plate connecting it to the end of the wrist. To reach such a trajectory, the base 106 of the robot 102 might be moved or a different end effector 112 with a different guide tube attachment might be exchanged with the working end effector. Both of these solutions may be time consuming and cumbersome.

As best seen in FIGS. 14A and 14B, if the array 908 is configured such that one or more of the markers 918A-918D are not in a fixed position and instead, one or more of the markers 918A-918D can be adjusted, swiveled, pivoted, or moved, the robot 102 can provide updated information about the object being tracked without disrupting the detection and tracking process. For example, one of the markers 918A-918D may be fixed in position and the other markers 918A-918D may be moveable; two of the markers 918A-918D may be fixed in position and the other markers 918A-918D may be moveable; three of the markers 918A-918D may be fixed in position and the other marker 918A-918D may be moveable; or all of the markers 918A-918D may be moveable.

In the embodiment shown in FIGS. 14A and 14B, markers 918A, 918 B are rigidly connected directly to a base 906 of the end-effector 912, and markers 918C, 918D are rigidly connected to the tube 914. Similar to array 612, array 908 may be provided to attach the markers 918A-918D to the end-effector 912, instrument 608, or other object to be tracked. In this case, however, the array 908 is comprised of a plurality of separate components. For example, markers 918A, 918B may be connected to the base 906 with a first array 908A, and markers 918C, 918D may be connected to the guide tube 914 with a second array 908B. Marker 918A may be affixed to a first end of the first array 908A and marker 918B may be separated a linear distance and affixed to a second end of the first array 908A. While first array 908 is substantially linear, second array 908B has a bent or V-shaped configuration, with respective root ends, connected to the guide tube 914, and diverging therefrom to distal ends in a V-shape with marker 918C at one distal end and marker 918D at the other distal end. Although specific configurations are exemplified herein, it will be appreciated that other asymmetric designs including different numbers and types of arrays 908A, 908B and different arrangements, numbers, and types of markers 918A-918D are contemplated.

The guide tube 914 may be moveable, swivelable, or pivotable relative to the base 906, for example, across a hinge 920 or other connector to the base 906. Thus, markers 918C, 918D are moveable such that when the guide tube 914 pivots, swivels, or moves, markers 918C, 918D also pivot, swivel, or move. As best seen in FIG. 14A, guide tube 914 has a longitudinal axis 916 which is aligned in a substantially normal or vertical orientation such that markers 918A-918D have a first configuration. Turning now to FIG. 14B, the guide tube 914 is pivoted, swiveled, or moved such that the longitudinal axis 916 is now angled relative to the vertical orientation such that markers 918A-918D have a second configuration, different from the first configuration.

In contrast to the embodiment described for FIGS. 14A-14D, if a swivel existed between the guide tube 914 and the arm 104 (e.g., the wrist attachment) with all four markers 918A-918D remaining attached rigidly to the guide tube 914 and this swivel was adjusted by the user, the robotic system 100, 300, 600 would not be able to automatically detect that the guide tube 914 orientation had changed. The robotic system 100, 300, 600 would track the positions of the marker array 908 and would calculate incorrect robot axis moves assuming the guide tube 914 was attached to the wrist (the robot arm 104) in the previous orientation. By keeping one or more markers 918A-918D (e.g., two markers 918C, 918D) rigidly on the tube 914 and one or more markers 918A-918D (e.g., two markers 918A, 918B) across the swivel, automatic detection of the new position becomes possible and correct robot moves are calculated based on the detection of a new tool or end-effector 112, 912 on the end of the robot arm 104.

One or more of the markers 918A-918D are configured to be moved, pivoted, swiveled, or the like according to any suitable means. For example, the markers 918A-918D may be moved by a hinge 920, such as a clamp, spring, lever, slide, toggle, or the like, or any other suitable mechanism for moving the markers 918A-918D individually or in combination, moving the arrays 908A, 908B individually or in combination, moving any portion of the end-effector 912 relative to another portion, or moving any portion of the tool 608 relative to another portion.

As shown in FIGS. 14A and 14B, the array 908 and guide tube 914 may become reconfigurable by simply loosening the clamp or hinge 920, moving part of the array 908A, 908B relative to the other part 908A, 908B, and retightening the hinge 920 such that the guide tube 914 is oriented in a different position. For example, two markers 918C, 918D may be rigidly interconnected with the tube 914 and two markers 918A, 918B may be rigidly interconnected across the hinge 920 to the base 906 of the end-effector 912 that attaches to the robot arm 104. The hinge 920 may be in the form of a clamp, such as a wing nut or the like, which can be loosened and retightened to allow the user to quickly switch between the first configuration (FIG. 14A) and the second configuration (FIG. 14B).

The cameras 200, 326 detect the markers 918A-918D, for example, in one of the templates identified in FIGS. 14C and 14D. If the array 908 is in the first configuration (FIG. 14A) and tracking cameras 200, 326 detect the markers 918A-918D, then the tracked markers match Array Template 1 as shown in FIG. 14C. If the array 908 is the second configuration (FIG. 14B) and tracking cameras 200, 326 detect the same markers 918A-918D, then the tracked markers match Array Template 2 as shown in FIG. 14D. Array Template 1 and Array Template 2 are recognized by the system 100, 300, 600 as two distinct tools, each with its own uniquely defined spatial relationship between guide tube 914, markers 918A-918D, and robot attachment. The user could therefore adjust the position of the end-effector 912 between the first and second configurations without notifying the system 100, 300, 600 of the change and the system 100, 300, 600 would appropriately adjust the movements of the robot 102 to stay on trajectory.

In this embodiment, there are two assembly positions in which the marker array matches unique templates that allow the system 100, 300, 600 to recognize the assembly as two different tools or two different end effectors. In any position of the swivel between or outside of these two positions (namely, Array Template 1 and Array Template 2 shown in FIGS. 14C and 14D, respectively), the markers 918A-918D would not match any template and the system 100, 300, 600 would not detect any array present despite individual markers 918A-918D being detected by cameras 200, 326, with the result being the same as if the markers 918A-918D were temporarily blocked from view of the cameras 200, 326. It will be appreciated that other array templates may exist for other configurations, for example, identifying different instruments 608 or other end-effectors 112, 912, etc.

In the embodiment described, two discrete assembly positions are shown in FIGS. 14A and 14B. It will be appreciated, however, that there could be multiple discrete positions on a swivel joint, linear joint, combination of swivel and linear joints, pegboard, or other assembly where unique marker templates may be created by adjusting the position of one or more markers 918A-918D of the array relative to the others, with each discrete position matching a particular template and defining a unique tool 608 or end-effector 112, 912 with different known attributes. In addition, although exemplified for end effector 912, it will be appreciated that moveable and fixed markers 918A-918D may be used with any suitable instrument 608 or other object to be tracked.

When using an external 3D tracking system 100, 300, 600 to track a full rigid body array of three or more markers attached to a robot's end effector 112 (for example, as depicted in FIGS. 13A and 13B), it is possible to directly track or to calculate the 3D position of every section of the robot 102 in the coordinate system of the cameras 200, 326. The geometric orientations of joints relative to the tracker are known by design, and the linear or angular positions of joints are known from encoders for each motor of the robot 102, fully defining the 3D positions of all of the moving parts from the end effector 112 to the base 116. Similarly, if a tracker were mounted on the base 106 of the robot 102 (not shown), it is likewise possible to track or calculate the 3D position of every section of the robot 102 from base 106 to end effector 112 based on known joint geometry and joint positions from each motor's encoder.

In some situations, it may be desirable to track the positions of all segments of the robot 102 from fewer than three markers 118 rigidly attached to the end effector 112. Specifically, if a tool 608 is introduced into the guide tube 114, it may be desirable to track full rigid body motion of the robot 902 with only one additional marker 118 being tracked.

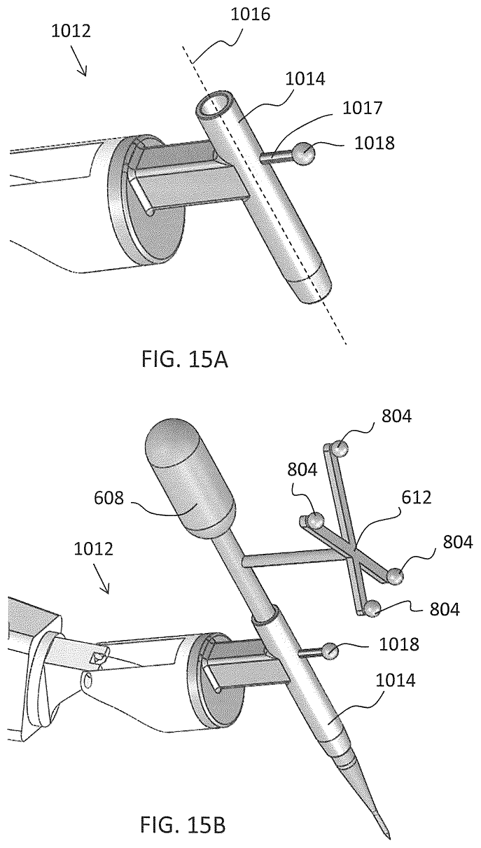

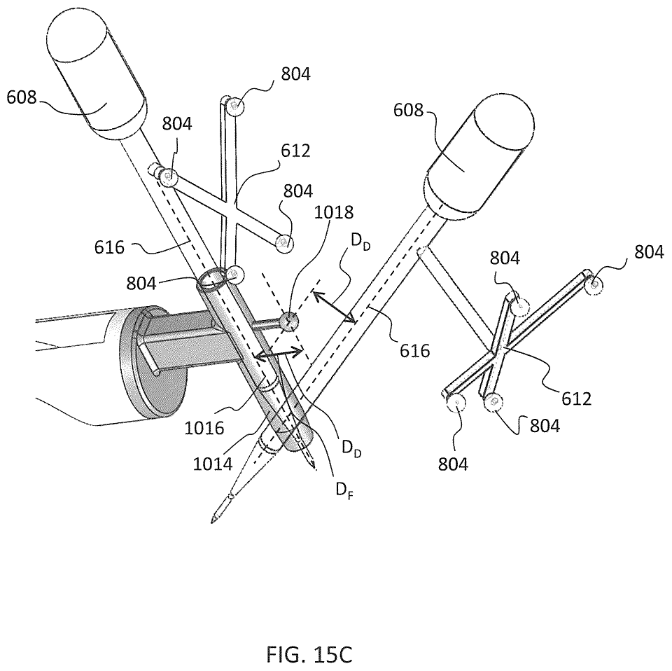

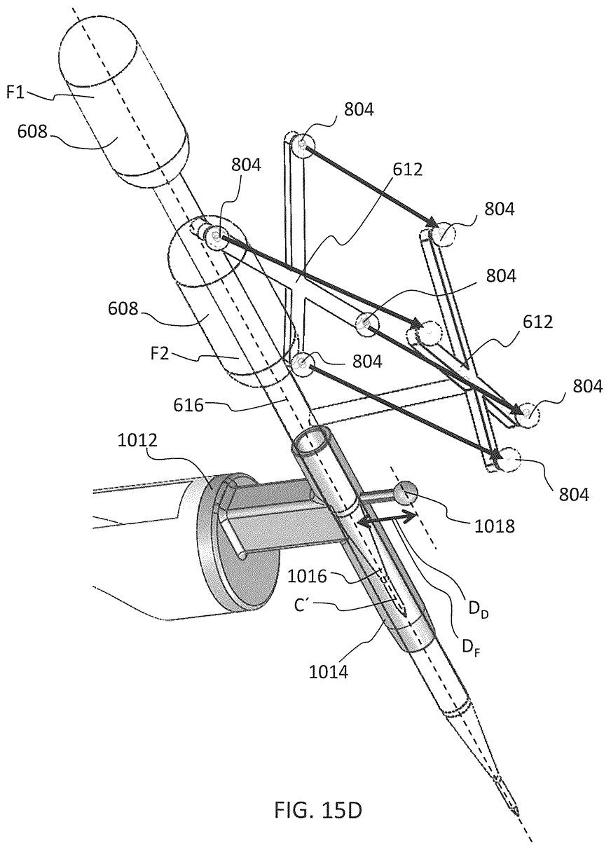

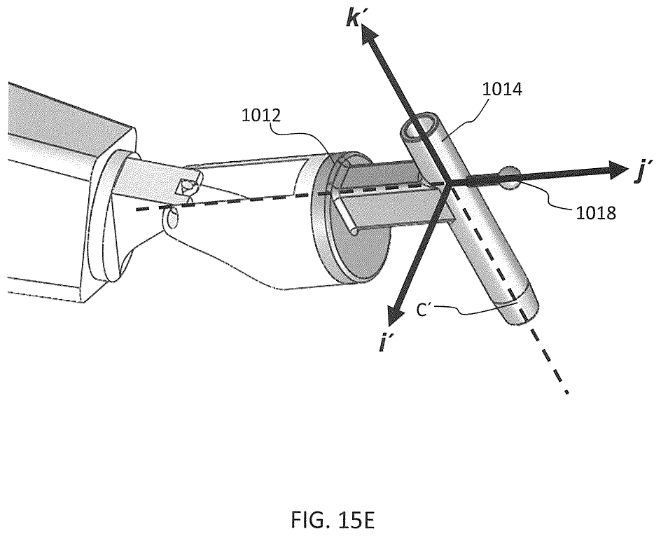

Turning now to FIGS. 15A-15E, an alternative version of an end-effector 1012 having only a single tracking marker 1018 is shown. End-effector 1012 may be similar to the other end-effectors described herein, and may include a guide tube 1014 extending along a longitudinal axis 1016. A single tracking marker 1018, similar to the other tracking markers described herein, may be rigidly affixed to the guide tube 1014. This single marker 1018 can serve the purpose of adding missing degrees of freedom to allow full rigid body tracking and/or can serve the purpose of acting as a surveillance marker to ensure that assumptions about robot and camera positioning are valid.

The single tracking marker 1018 may be attached to the robotic end effector 1012 as a rigid extension to the end effector 1012 that protrudes in any convenient direction and does not obstruct the surgeon's view. The tracking marker 1018 may be affixed to the guide tube 1014 or any other suitable location of on the end-effector 1012. When affixed to the guide tube 1014, the tracking marker 1018 may be positioned at a location between first and second ends of the guide tube 1014. For example, in FIG. 15A, the single tracking marker 1018 is shown as a reflective sphere mounted on the end of a narrow shaft 1017 that extends forward from the guide tube 1014 and is positioned longitudinally above a mid-point of the guide tube 1014 and below the entry of the guide tube 1014. This position allows the marker 1018 to be generally visible by cameras 200, 326 but also would not obstruct vision of the surgeon 120 or collide with other tools or objects in the vicinity of surgery. In addition, the guide tube 1014 with the marker 1018 in this position is designed for the marker array on any tool 608 introduced into the guide tube 1014 to be visible at the same time as the single marker 1018 on the guide tube 1014 is visible.

As shown in FIG. 15B, when a snugly fitting tool or instrument 608 is placed within the guide tube 1014, the instrument 608 becomes mechanically constrained in 4 of 6 degrees of freedom. That is, the instrument 608 cannot be rotated in any direction except about the longitudinal axis 1016 of the guide tube 1014 and the instrument 608 cannot be translated in any direction except along the longitudinal axis 1016 of the guide tube 1014. In other words, the instrument 608 can only be translated along and rotated about the centerline of the guide tube 1014. If two more parameters are known, such as (1) an angle of rotation about the longitudinal axis 1016 of the guide tube 1014; and (2) a position along the guide tube 1014, then the position of the end effector 1012 in the camera coordinate system becomes fully defined.

Referring now to FIG. 15C, the system 100, 300, 600 should be able to know when a tool 608 is actually positioned inside of the guide tube 1014 and is not instead outside of the guide tube 1014 and just somewhere in view of the cameras 200, 326. The tool 608 has a longitudinal axis or centerline 616 and an array 612 with a plurality of tracked markers 804. The rigid body calculations may be used to determine where the centerline 616 of the tool 608 is located in the camera coordinate system based on the tracked position of the array 612 on the tool 608.

The fixed normal (perpendicular) distance D_{F} from the single marker 1018 to the centerline or longitudinal axis 1016 of the guide tube 1014 is fixed and is known geometrically, and the position of the single marker 1018 can be tracked. Therefore, when a detected distance D_{D} from tool centerline 616 to single marker 1018 matches the known fixed distance D_{F} from the guide tube centerline 1016 to the single marker 1018, it can be determined that the tool 608 is either within the guide tube 1014 (centerlines 616, 1016 of tool 608 and guide tube 1014 coincident) or happens to be at some point in the locus of possible positions where this distance D_{D} matches the fixed distance D_{F}. For example, in FIG. 15C, the normal detected distance D_{D} from tool centerline 616 to the single marker 1018 matches the fixed distance D_{F} from guide tube centerline 1016 to the single marker 1018 in both frames of data (tracked marker coordinates) represented by the transparent tool 608 in two positions, and thus, additional considerations may be needed to determine when the tool 608 is located in the guide tube 1014.

Turning now to FIG. 15D, programmed logic can be used to look for frames of tracking data in which the detected distance D_{D} from tool centerline 616 to single marker 1018 remains fixed at the correct length despite the tool 608 moving in space by more than some minimum distance relative to the single sphere 1018 to satisfy the condition that the tool 608 is moving within the guide tube 1014. For example, a first frame F1 may be detected with the tool 608 in a first position and a second frame F2 may be detected with the tool 608 in a second position (namely, moved linearly with respect to the first position). The markers 804 on the tool array 612 may move by more than a given amount (e.g., more than 5 mm total) from the first frame F1 to the second frame F2. Even with this movement, the detected distance D_{D} from the tool centerline vector C′ to the single marker 1018 is substantially identical in both the first frame F1 and the second frame F2.

Logistically, the surgeon 120 or user could place the tool 608 within the guide tube 1014 and slightly rotate it or slide it down into the guide tube 1014 and the system 100, 300, 600 would be able to detect that the tool 608 is within the guide tube 1014 from tracking of the five markers (four markers 804 on tool 608 plus single marker 1018 on guide tube 1014). Knowing that the tool 608 is within the guide tube 1014, all 6 degrees of freedom may be calculated that define the position and orientation of the robotic end effector 1012 in space. Without the single marker 1018, even if it is known with certainty that the tool 608 is within the guide tube 1014, it is unknown where the guide tube 1014 is located along the tool's centerline vector C′ and how the guide tube 1014 is rotated relative to the centerline vector C′.

With emphasis on FIG. 15E, the presence of the single marker 1018 being tracked as well as the four markers 804 on the tool 608, it is possible to construct the centerline vector C′ of the guide tube 1014 and tool 608 and the normal vector through the single marker 1018 and through the centerline vector C′. This normal vector has an orientation that is in a known orientation relative to the forearm of the robot distal to the wrist (in this example, oriented parallel to that segment) and intersects the centerline vector C′ at a specific fixed position. For convenience, three mutually orthogonal vectors k′, j′, i′ can be constructed, as shown in FIG. 15E, defining rigid body position and orientation of the guide tube 1014. One of the three mutually orthogonal vectors k′ is constructed from the centerline vector C′, the second vector j′ is constructed from the normal vector through the single marker 1018, and the third vector i′ is the vector cross product of the first and second vectors k′, j′. The robot's joint positions relative to these vectors k′, j′, i′ are known and fixed when all joints are at zero, and therefore rigid body calculations can be used to determine the location of any section of the robot relative to these vectors k′, j′, i′ when the robot is at a home position. During robot movement, if the positions of the tool markers 804 (while the tool 608 is in the guide tube 1014) and the position of the single marker 1018 are detected from the tracking system, and angles/linear positions of each joint are known from encoders, then position and orientation of any section of the robot can be determined.

In some embodiments, it may be useful to fix the orientation of the tool 608 relative to the guide tube 1014. For example, the end effector guide tube 1014 may be oriented in a particular position about its axis 1016 to allow machining or implant positioning. Although the orientation of anything attached to the tool 608 inserted into the guide tube 1014 is known from the tracked markers 804 on the tool 608, the rotational orientation of the guide tube 1014 itself in the camera coordinate system is unknown without the additional tracking marker 1018 (or multiple tracking markers in other embodiments) on the guide tube 1014. This marker 1018 provides essentially a “clock position” from −180° to +180° based on the orientation of the marker 1018 relative to the centerline vector C′. Thus, the single marker 1018 can provide additional degrees of freedom to allow full rigid body tracking and/or can act as a surveillance marker to ensure that assumptions about the robot and camera positioning are valid.

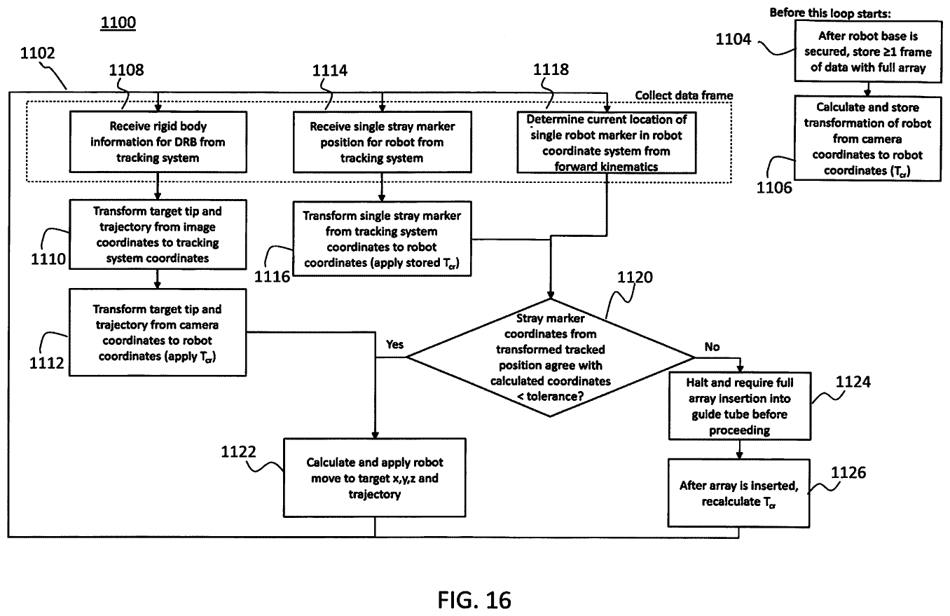

FIG. 16 is a block diagram of a method 1100 for navigating and moving the end-effector 1012 (or any other end-effector described herein) of the robot 102 to a desired target trajectory. Another use of the single marker 1018 on the robotic end effector 1012 or guide tube 1014 is as part of the method 1100 enabling the automated safe movement of the robot 102 without a full tracking array attached to the robot 102. This method 1100 functions when the tracking cameras 200, 326 do not move relative to the robot 102 (i.e., they are in a fixed position), the tracking system's coordinate system and robot's coordinate system are co-registered, and the robot 102 is calibrated such that the position and orientation of the guide tube 1014 can be accurately determined in the robot's Cartesian coordinate system based only on the encoded positions of each robotic axis.

For this method 1100, the coordinate systems of the tracker and the robot must be co-registered, meaning that the coordinate transformation from the tracking system's Cartesian coordinate system to the robot's Cartesian coordinate system is needed. For convenience, this coordinate transformation can be a 4×4 matrix of translations and rotations that is well known in the field of robotics. This transformation will be termed Tcr to refer to “transformation—camera to robot”. Once this transformation is known, any new frame of tracking data, which is received as x,y,z coordinates in vector form for each tracked marker, can be multiplied by the 4×4 matrix and the resulting x,y,z coordinates will be in the robot's coordinate system. To obtain Tcr, a full tracking array on the robot is tracked while it is rigidly attached to the robot at a location that is known in the robot's coordinate system, then known rigid body methods are used to calculate the transformation of coordinates. It should be evident that any tool 608 inserted into the guide tube 1014 of the robot 102 can provide the same rigid body information as a rigidly attached array when the additional marker 1018 is also read. That is, the tool 608 need only be inserted to any position within the guide tube 1014 and at any rotation within the guide tube 1014, not to a fixed position and orientation. Thus, it is possible to determine Tcr by inserting any tool 608 with a tracking array 612 into the guide tube 1014 and reading the tool's array 612 plus the single marker 1018 of the guide tube 1014 while at the same time determining from the encoders on each axis the current location of the guide tube 1014 in the robot's coordinate system.

Logic for navigating and moving the robot 102 to a target trajectory is provided in the method 1100 of FIG. 16. Before entering the loop 1102, it is assumed that the transformation Tcr was previously stored. Thus, before entering loop 1102, in step 1104, after the robot base 106 is secured, greater than or equal to one frame of tracking data of a tool inserted in the guide tube while the robot is static is stored; and in step 1106, the transformation of robot guide tube position from camera coordinates to robot coordinates Tcr is calculated from this static data and previous calibration data. Tcr should remain valid as long as the cameras 200, 326 do not move relative to the robot 102. If the cameras 200, 326 move relative to the robot 102, and Tcr needs to be re-obtained, the system 100, 300, 600 can be made to prompt the user to insert a tool 608 into the guide tube 1014 and then automatically perform the necessary calculations.

In the flowchart of method 1100, each frame of data collected consists of the tracked position of the DRB 1404 on the patient 210, the tracked position of the single marker 1018 on the end effector 1014, and a snapshot of the positions of each robotic axis. From the positions of the robot's axes, the location of the single marker 1018 on the end effector 1012 is calculated. This calculated position is compared to the actual position of the marker 1018 as recorded from the tracking system. If the values agree, it can be assured that the robot 102 is in a known location. The transformation Tcr is applied to the tracked position of the DRB 1404 so that the target for the robot 102 can be provided in terms of the robot's coordinate system. The robot 102 can then be commanded to move to reach the target.

After steps 1104, 1106, loop 1102 includes step 1108 receiving rigid body information for DRB 1404 from the tracking system; step 1110 transforming target tip and trajectory from image coordinates to tracking system coordinates; and step 1112 transforming target tip and trajectory from camera coordinates to robot coordinates (apply Tcr). Loop 1102 further includes step 1114 receiving a single stray marker position for robot from tracking system; and step 1116 transforming the single stray marker from tracking system coordinates to robot coordinates (apply stored Tcr). Loop 1102 also includes step 1118 determining current location of the single robot marker 1018 in the robot coordinate system from forward kinematics. The information from steps 1116 and 1118 is used to determine step 1120 whether the stray marker coordinates from transformed tracked position agree with the calculated coordinates being less than a given tolerance. If yes, proceed to step 1122, calculate and apply robot move to target x, y, z and trajectory. If no, proceed to step 1124, halt and require full array insertion into guide tube 1014 before proceeding; step 1126 after array is inserted, recalculate Tcr; and then proceed to repeat steps 1108, 1114, and 1118.

This method 1100 has advantages over a method in which the continuous monitoring of the single marker 1018 to verify the location is omitted. Without the single marker 1018, it would still be possible to determine the position of the end effector 1012 using Tcr and to send the end-effector 1012 to a target location but it would not be possible to verify that the robot 102 was actually in the expected location. For example, if the cameras 200, 326 had been bumped and Tcr was no longer valid, the robot 102 would move to an erroneous location. For this reason, the single marker 1018 provides value with regard to safety.

For a given fixed position of the robot 102, it is theoretically possible to move the tracking cameras 200, 326 to a new location in which the single tracked marker 1018 remains unmoved since it is a single point, not an array. In such a case, the system 100, 300, 600 would not detect any error since there would be agreement in the calculated and tracked locations of the single marker 1018. However, once the robot's axes caused the guide tube 1012 to move to a new location, the calculated and tracked positions would disagree and the safety check would be effective.

The term “surveillance marker” may be used, for example, in reference to a single marker that is in a fixed location relative to the DRB 1404. In this instance, if the DRB 1404 is bumped or otherwise dislodged, the relative location of the surveillance marker changes and the surgeon 120 can be alerted that there may be a problem with navigation. Similarly, in the embodiments described herein, with a single marker 1018 on the robot's guide tube 1014, the system 100, 300, 600 can continuously check whether the cameras 200, 326 have moved relative to the robot 102. If registration of the tracking system's coordinate system to the robot's coordinate system is lost, such as by cameras 200, 326 being bumped or malfunctioning or by the robot malfunctioning, the system 100, 300, 600 can alert the user and corrections can be made. Thus, this single marker 1018 can also be thought of as a surveillance marker for the robot 102.

It should be clear that with a full array permanently mounted on the robot 102 (e.g., the plurality of tracking markers 702 on end-effector 602 shown in FIGS. 7A-7C) such functionality of a single marker 1018 as a robot surveillance marker is not needed because it is not required that the cameras 200, 326 be in a fixed position relative to the robot 102, and Tcr is updated at each frame based on the tracked position of the robot 102. Reasons to use a single marker 1018 instead of a full array are that the full array is more bulky and obtrusive, thereby blocking the surgeon's view and access to the surgical field 208 more than a single marker 1018, and line of sight to a full array is more easily blocked than line of sight to a single marker 1018.

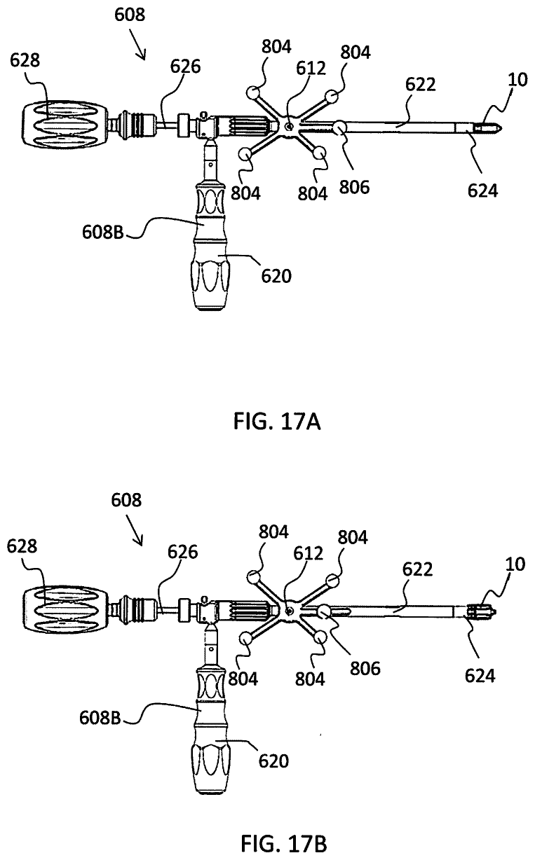

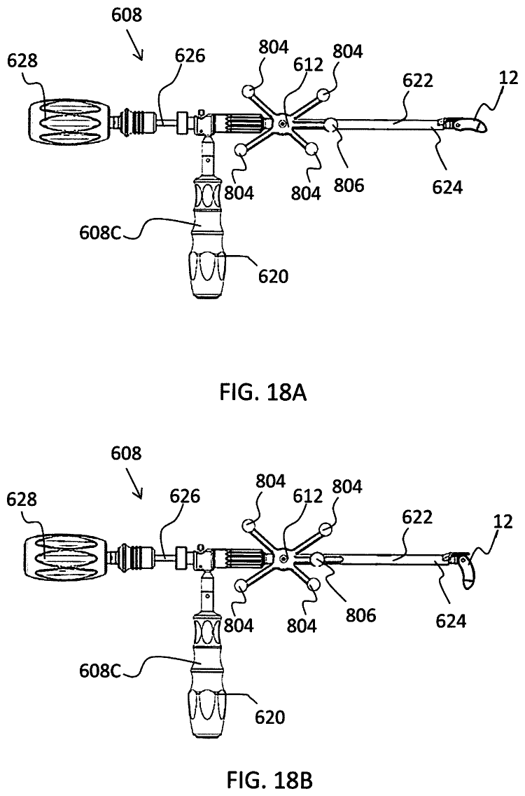

Turning now to FIGS. 17A-17B and 18A-18B, instruments 608, such as implant holders 608B, 608C, are depicted which include both fixed and moveable tracking markers 804, 806. The implant holders 608B, 608C may have a handle 620 and an outer shaft 622 extending from the handle 620. The shaft 622 may be positioned substantially perpendicular to the handle 620, as shown, or in any other suitable orientation. An inner shaft 626 may extend through the outer shaft 622 with a knob 628 at one end. Implant 10, 12 connects to the shaft 622, at the other end, at tip 624 of the implant holder 608B, 608C using typical connection mechanisms known to those of skill in the art. The knob 628 may be rotated, for example, to expand or articulate the implant 10, 12. U.S. Pat. Nos. 8,709,086 and 8,491,659, which are incorporated by reference herein, describe expandable fusion devices and methods of installation.

When tracking the tool 608, such as implant holder 608B, 608C, the tracking array 612 may contain a combination of fixed markers 804 and one or more moveable markers 806 which make up the array 612 or is otherwise attached to the implant holder 608B, 608C. The navigation array 612 may include at least one or more (e.g., at least two) fixed position markers 804, which are positioned with a known location relative to the implant holder instrument 608B, 608C. These fixed markers 804 would not be able to move in any orientation relative to the instrument geometry and would be useful in defining where the instrument 608 is in space. In addition, at least one marker 806 is present which can be attached to the array 612 or the instrument itself which is capable of moving within a pre-determined boundary (e.g., sliding, rotating, etc.) relative to the fixed markers 804. The system 100, 300, 600 (e.g., the software) correlates the position of the moveable marker 806 to a particular position, orientation, or other attribute of the implant 10 (such as height of an expandable interbody spacer shown in FIGS. 17A-17B or angle of an articulating interbody spacer shown in FIGS. 18A-18B). Thus, the system and/or the user can determine the height or angle of the implant 10, 12 based on the location of the moveable marker 806.

In the embodiment shown in FIGS. 17A-17B, four fixed markers 804 are used to define the implant holder 608B and a fifth moveable marker 806 is able to slide within a pre-determined path to provide feedback on the implant height (e.g., a contracted position or an expanded position). FIG. 17A shows the expandable spacer 10 at its initial height, and FIG. 17B shows the spacer 10 in the expanded state with the moveable marker 806 translated to a different position. In this case, the moveable marker 806 moves closer to the fixed markers 804 when the implant 10 is expanded, although it is contemplated that this movement may be reversed or otherwise different. The amount of linear translation of the marker 806 would correspond to the height of the implant 10. Although only two positions are shown, it would be possible to have this as a continuous function whereby any given expansion height could be correlated to a specific position of the moveable marker 806.

Turning now to FIGS. 18A-18B, four fixed markers 804 are used to define the implant holder 608C and a fifth, moveable marker 806 is configured to slide within a pre-determined path to provide feedback on the implant articulation angle. FIG. 18A shows the articulating spacer 12 at its initial linear state, and FIG. 18B shows the spacer 12 in an articulated state at some offset angle with the moveable marker 806 translated to a different position. The amount of linear translation of the marker 806 would correspond to the articulation angle of the implant 12. Although only two positions are shown, it would be possible to have this as a continuous function whereby any given articulation angle could be correlated to a specific position of the moveable marker 806.

In these embodiments, the moveable marker 806 slides continuously to provide feedback about an attribute of the implant 10, 12 based on position. It is also contemplated that there may be discreet positions that the moveable marker 806 must be in which would also be able to provide further information about an implant attribute. In this case, each discreet configuration of all markers 804, 806 correlates to a specific geometry of the implant holder 608B, 608C and the implant 10, 12 in a specific orientation or at a specific height. In addition, any motion of the moveable marker 806 could be used for other variable attributes of any other type of navigated implant.

Although depicted and described with respect to linear movement of the moveable marker 806, the moveable marker 806 should not be limited to just sliding as there may be applications where rotation of the marker 806 or other movements could be useful to provide information about the implant 10, 12. Any relative change in position between the set of fixed markers 804 and the moveable marker 806 could be relevant information for the implant 10, 12 or other device. In addition, although expandable and articulating implants 10, 12 are exemplified, the instrument 608 could work with other medical devices and materials, such as spacers, cages, plates, fasteners, nails, screws, rods, pins, wire structures, sutures, anchor clips, staples, stents, bone grafts, biologics, or the like.

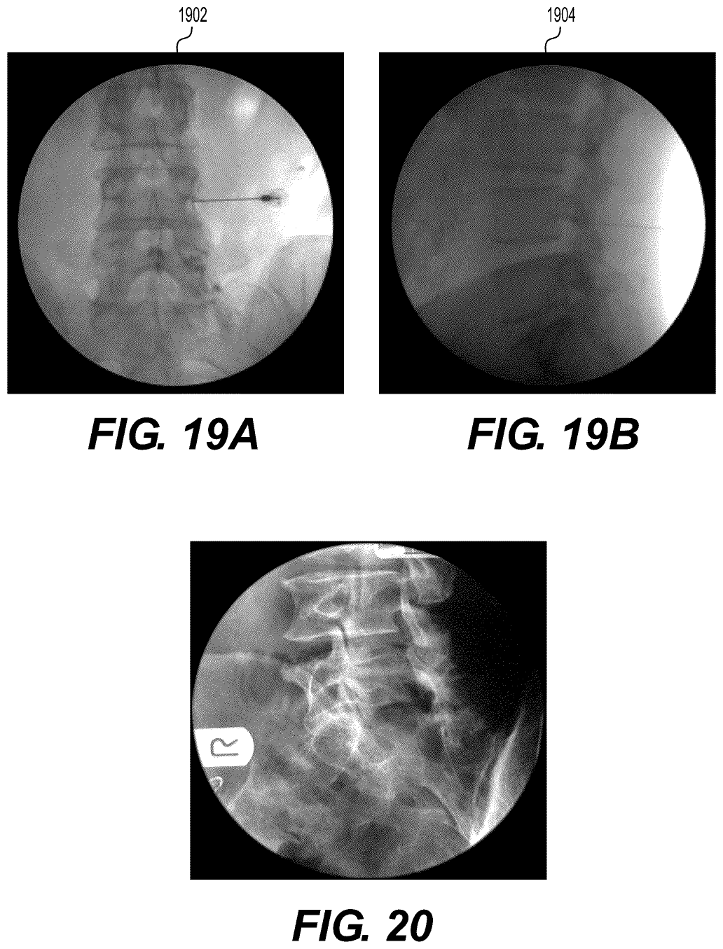

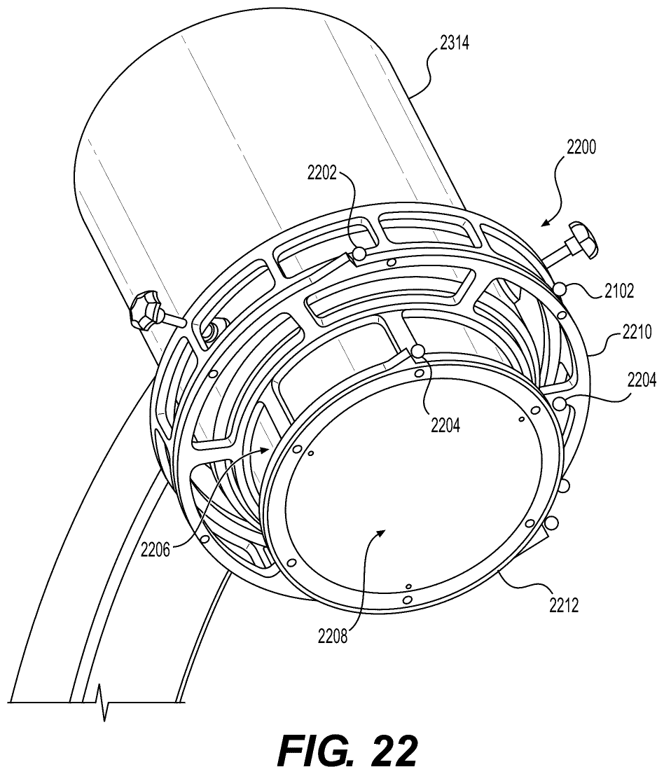

One aspect of the present invention related to determining the 3-dimensional position of an imaging arm of an imaging device for taking optimal images of a vertebral body will now be explained with reference to FIGS. 19-27.

Most conventional systems do not have navigation capabilities and rely on users to position the C-arm. A few systems may have some navigation functions that allow a user to return to the previously stored position. In other words, existing systems may have the capability to let the user know where the imaging system may have been in the past. By contrast, the present invention as described with FIGS. 19-27 proposes to let the user know where the imaging system will need to be in the future to take optimal images.

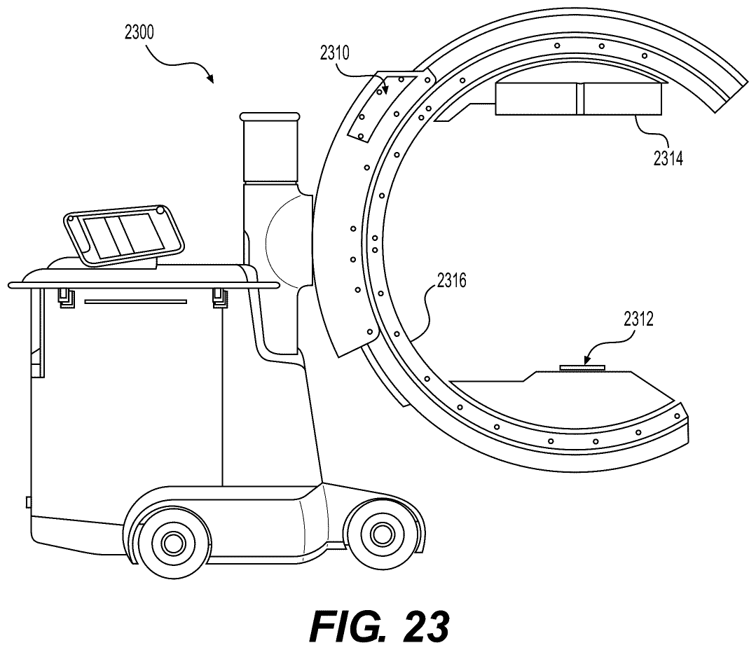

FIG. 23 is an example of an x-ray imaging device 2300 having an automatic positioning capability with respect to the 3D position and orientation of its C-arm 2316 according to one aspect of the present invention. The imaging device 2300 includes a detector panel assembly 2314 containing a sensor array (not shown) for receiving x-ray transmission from an x-ray source 2312. The imaging device 2300 is more fully described in U.S. Pat. No. 1,044,8910 assigned to the applicant of the present invention, which is incorporate herein by reference. The imaging device 2300 is capable of communicating with the surgical robot system 300 through the connector panel 320 by a physical I/O cable or wirelessly through well-known wireless transmission methods including WiFi, Bluetooth and the like.

Unlike the imaging system 2300, which does not require a calibration ring, manually operated C-arms such as 1308 typically will have a calibration ring 2200 mounted to the detector panel assembly 1316 as shown in FIG. 12A. The calibration ring 2200 includes two spaced apart rings 2210,2212, each with a planar surface. Each planar surface contains a plurality of radiopaque markers 2206,2208 that are spaced apart from each other in a selected pattern. Two sets of a plurality of circumferentially spaced optical markers 2202,2204 are also mounted to the rings 2210,2212. The radiopaque markers 2206,2208 are used to perform an initial registration of the imaging device 1308 (i.e., mapping of the C-arm position and orientation relative to the patient from the imaging space to the camera coordinate system) so that the tracking subsystem 532 can track the position and orientation of the C-arm 1308 during the surgical procedure. For au automatically navigated imaging system 2300, a calibration ring is unnecessary and tracking and navigation of the system can be done with optical markers 2310 or the encoders that are positioned in every moving part of the system. The encoders can be used to mark the relative location and orientation of the C-arm 2316 at any time in use.

Once initial registration has been performed, the cameras 326 of the tracking subsystem 532 can continuously track the C-arm 2316 position and orientation through the optical markers 2202, 2204, and optionally through the markers 2310 on the C-arm 2316 during the surgical procedure.

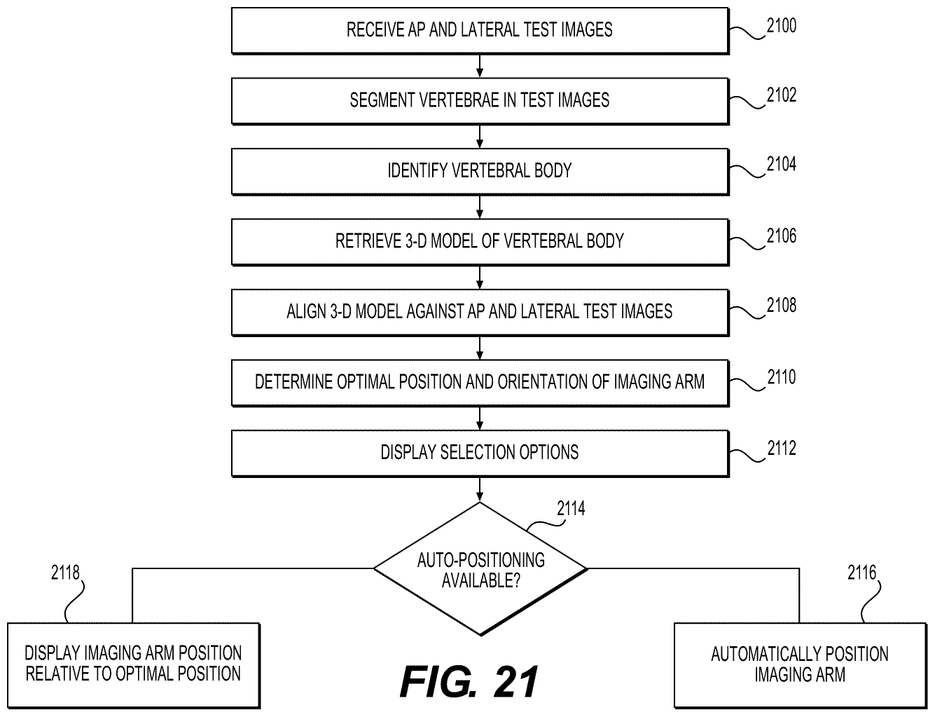

FIG. 21 illustrates a flowchart of a method of determining the 3-dimensional (3D) position and orientation of an imaging device for each vertebral level for taking optimal AP and lateral images so that only one set of images are needed. The processing steps in FIG. 21 can be performed by an image control module 409 in the computer 408, processor of the imaging device 2300 itself, other remotely located processors or a combination thereof. In one embodiment, the image control module 409 includes computer executable code stored in a memory 410.

In step 2100, a user (typically an x-ray technician in the operating room) positions the imaging device 2300 around a patient table (not shown) such that the patient lying on the table is position inside the C-arm 2316. Once the imaging device 2300 is positioned, a pair of x-ray images (one AP image and one lateral image) are taken by the user without regard to how accurately or optimally the C-arm 2316 is positioned so long as the vertebral levels of interest are included. A typical AP image 2602 and lateral image 2604 are shown in FIG. 26. Once the two images are taken, they are received and stored by the computer 408.

Along with the images, the computer 408 also receives and stores the 3D position and orientation of the C-arm (e.g., 3D position and orientation of the imaging panel/intensifier or x-ray source of the C-arm, or both) for each of the two images 2602,2604.

In step 2102, the vertebral bodies of interest are segmented for later analysis. Segmentation is a process by which certain points or features on a body part such as a vertebral body are identified. It can be manual, semi-automatic or fully automatic. An illustration of segmented vertebral bodies is shown in FIG. 26. The semi-automatic or fully automatic segmentation methods for identifying the relevant points of the vertebral body are well-known in the art. For example, an open-source software program called “ITK-SNAP” (available at www.itksnap.net) may allow the user to interactively segment out each vertebral body.

Step 2102 may also identify the vertebral levels as part of the segmentation process. This identification process can be totally manual, which requires a user to identify each level. Alternatively, the identification process can also be semi-automatic or fully automatic. In a semi-automatic case, the user may identify at least one level and the remaining levels are automatically applied based on image processing. For example, once the user identifies one vertebral body as being L4 (as shown in FIG. 26), the computer 408 automatically identifies all other levels based on the lordotic angle of the segmented bodies, for example. As a double check, the computer 408 may ask the user to confirm that the automatically identified levels, either by semi-automatic or fully automatic process, are correct.

In step 2104, the computer 408 asks the user to identify which vertebral levels are of interest. The user then identifies them using a graphical user interface, for example by touching the displayed levels on a touch screen display device 304 (e.g., four levels from L1 to L4).

In step 2106, the computer 408 retrieves from a database a 3D model 2702 of the spine including the vertebral bodies of interest. The 3D model may be based on a statistical model which is not specific to any patient as most spines generally follow a standard pattern or it could be based on a specific patient in question from a 3D scan. Alternatively, the standard 3D model can be enhanced by patient specific data such as the lordotic and kyphotic angles which are derived from the images 2602,2604. The retrieved vertebral bodies 2702 are then scaled so that the size of the bodies are the same as those in the AP and lateral images. The scaling may be based on the segmentation information obtained from step 2102.

In step 2106, for each vertebral body of interest, the computer 408 performs an alignment of the retrieved 3D model of a selected vertebral body to the corresponding segmented vertebral body in the AP and lateral images 2602,2604. One method that may be used is a “fluoro-CT merge”, for example. One algorithm for the fluoro-CT merge can be found in an article entitled “Image-Assisted Navigation System for Spinal Surgery”, Applied Bionics and Biomechanics, Volume 2015, Article ID 478062, 9 pages, published May 28, 2015 (downloaded from http://dx.doi.org/10.1155/2015/478062), which is incorporated herein by reference. Essentially, the 3D vertebral model's position and orientation (including X, Y, Z, Yaw, Roll and Pitch) is adjusted by the computer 408 until an optimum alignment is achieved.

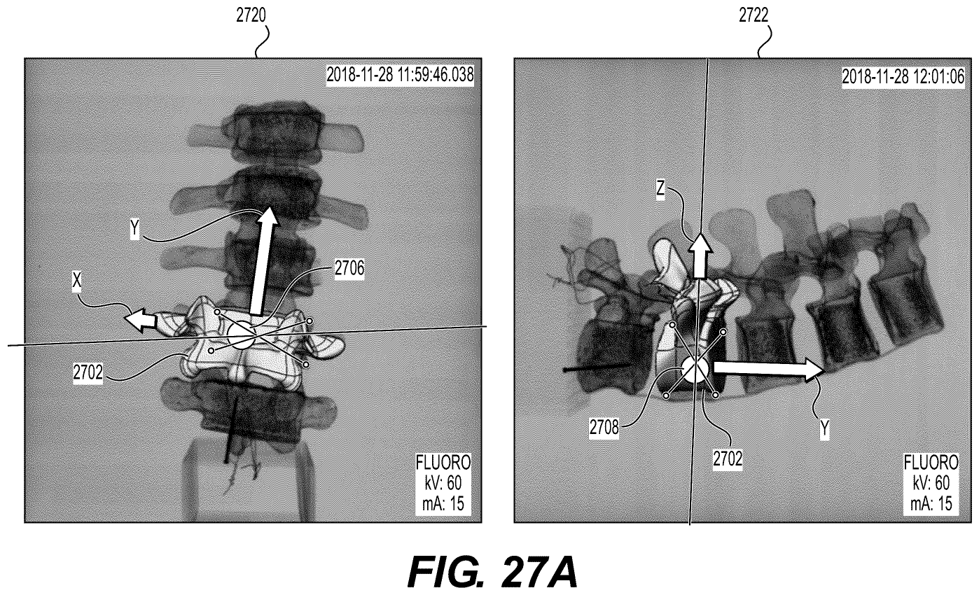

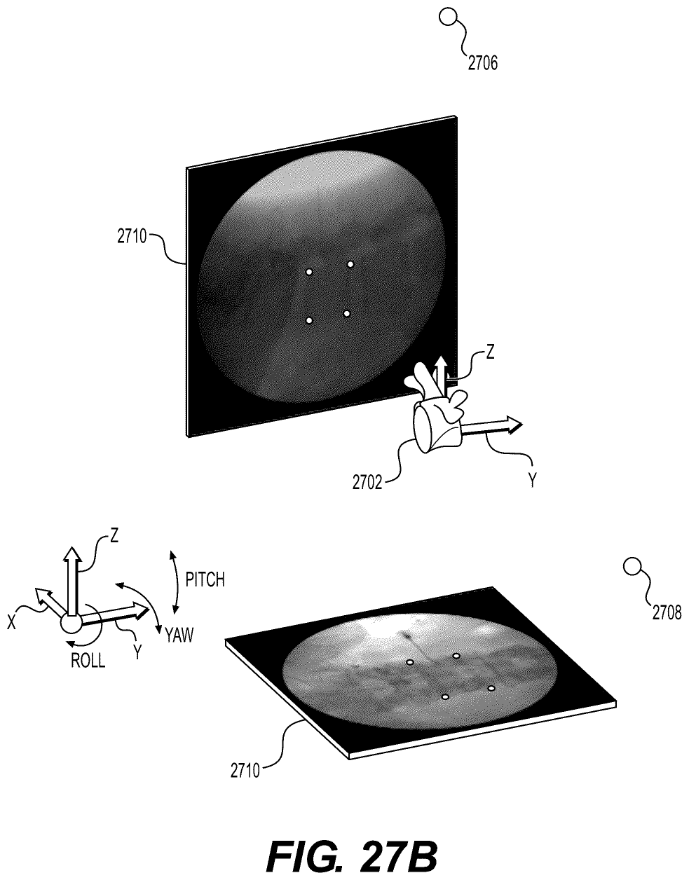

FIGS. 27A and 27B graphically illustrate the alignment method. FIG. 27B illustrates the lateral test image 2710 from an x-ray source 2708. The lateral test image 2710 and x-ray source 2706 respectively correspond to the detector panel 2710 in the detector panel assembly 2314 and x-ray source 2312 of the imaging device 2300 at the time the test image was taken. The AP test image 2710 and x-ray source 2706 respectively correspond to the detector panel 2710 and x-ray source 2312 of the imaging device 2300 at the time the test image was taken. As can be seen, the model vertebral body 2702 is scaled and manipulated until the body matches most closely aligns with the corresponding vertebral body in the AP and lateral images.

Step 2106 is repeated for each vertebral body of interest as identified in step 2104.

Then, in step 2110, based on the optimal 3D position and orientation of the vertebral body as determined in step 2108, the computer 408 determines the optimal C-arm 2316 orientation and position (e.g., 3D position and orientation of either the detector panel 2314 or the x-ray source 2312, or both) so as to center the vertebral body with perfect AP and lateral angles. Then, the determined optimal C-arm 2316 orientation and position for the vertebral body are stored in the memory 410.

This optimal C-arm 2316 orientation and position determination of step 2110 can be partially seen in FIG. 27A. FIG. 27A shows the model vertebral body 2702 which has been aligned with the corresponding vertebral body in the AP and lateral test images 2602,2604. As can be readily seen from the left screen shot 2720 showing a test AP image 2602, an optimum position for the C-arm 2316 would include rotating it clockwise by about 15 degrees and moving it down by about half a vertebral level to center the vertebral body. From the right screen shot 2722 showing a lateral test image 2604, an optimum position for the C-arm 2316 would include rotating it counter-clockwise by about 10 degrees and to move it left by about half a vertebral level to center the vertebral body in the image.

The optimal C-arm 2316 orientation and position of the vertebral body are then stored in the memory. In one embodiment, the orientation and position information for taking one of the two images are stored. Then, taking the other image is just a matter of rotating the C-arm 2316 by 90 degrees. In an alternative embodiment, the orientation and position information for taking both AP and lateral images are stored. If there are any additional levels that have not been processed, then steps 2106-2110 may be repeated.

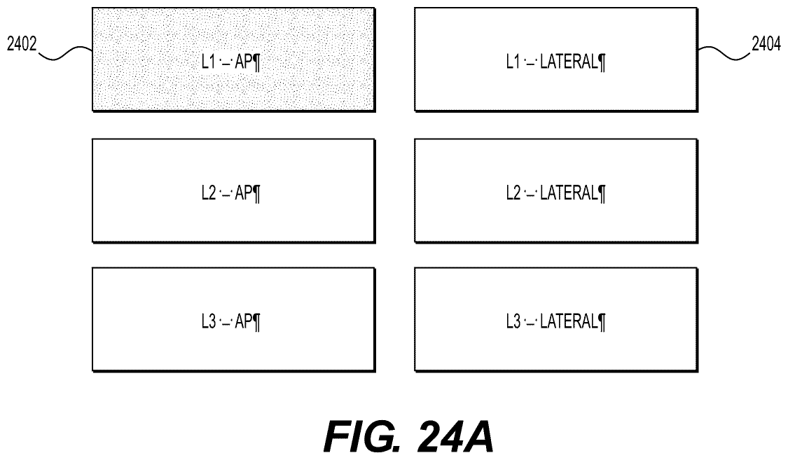

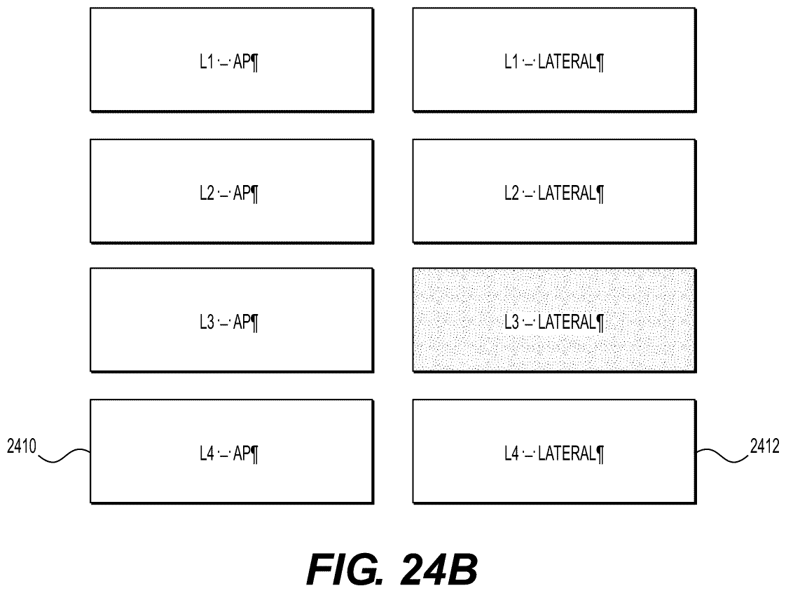

In step 2112, the computer 408 displays the available vertebral levels for optimal imaging for user selection in the display device 304, one example of which is illustrated in FIG. 24A. For each level, the display 304 displays two user input buttons 2402 and 2404. These buttons are used to position the imaging system 2300 to the ideal or optimal imaging position. The positioning can be either manual or automatic, depending on the image equipment being used. Button 2402 is for taking an AP image and box 2404 is for taking a lateral image. The user can select the image to be taken by an input device such as a mouse, touchscreen or keyboard. In one embodiment, the user can make the selection by touching the input button through a touch-sensitive screen of the display device 304.

In decision 2114, the computer 408 determine whether the imaging device 2300 has an automatic positioning capability. The automatic positioning capability allows the computer 408 to send position and orientation commands to move and rotate the C-arm 2316 of the imaging device 2300 in an optimal 3D position and orientation as determined in step 2110.

If the imaging device 2300 is determined to have such a capability, then control passes to step 2116. In step 2116, the computer 408 sends the optimal 3D position and orientation of the C-arm 2316 to the imaging device.

In one embodiment, the computer 408 sends an absolute position and orientation data to the imaging device 2300. This is possible if the imaging device 2300 knows its exact position within the operating room. In another embodiment, the computer 408 sends movement instructions that incrementally moves and positions the C-arm 2316 step by step. The computer 408 knows the relative position of the C-arm 2316 from the initial registration of the imaging device 2300 to the patient. From the registration data and the optical markers 2310 on the gantry, the computer 408 can track the relative location and orientation of the C-arm 2316 relative to the patient. From the tracking data and while the markers are being tracked, the computer can issue a series of incremental positioning commands to the imaging device 2300 until optimal C-arm 2316 position and orientation are reached.

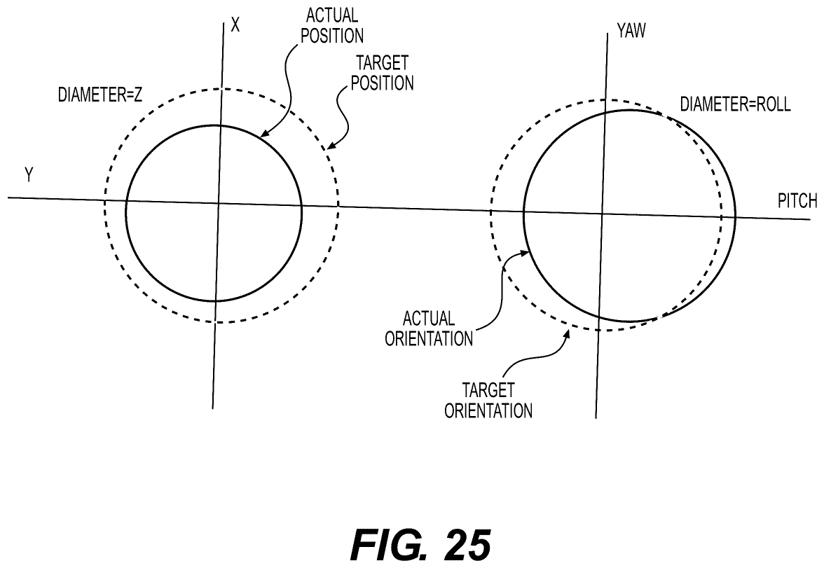

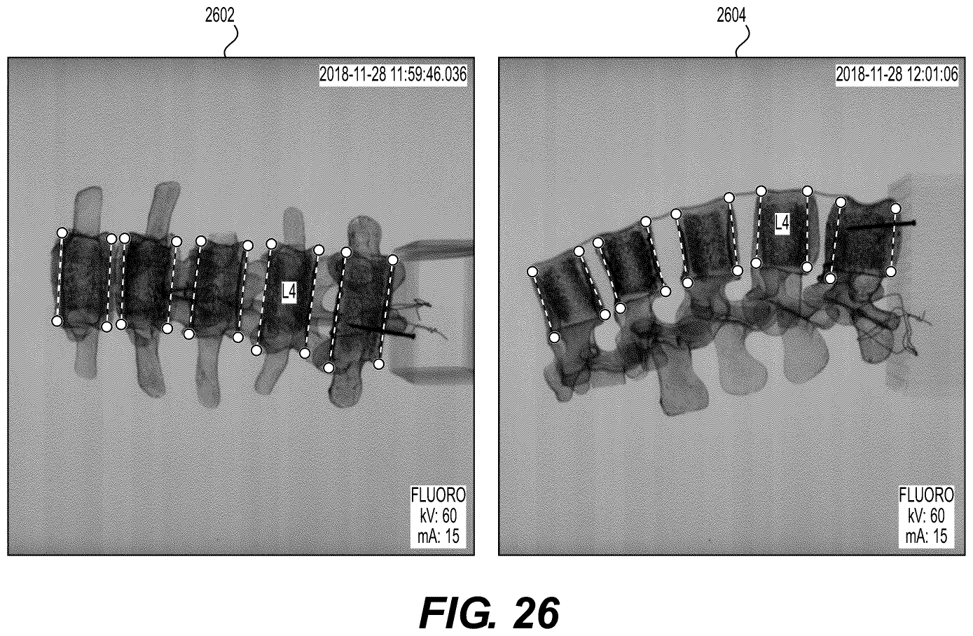

If the imaging device is determined not to have such an automatic positioning capability (such as imaging system 1304 of FIG. 12A) in step 2114, then control passes to step 2118. In step 2118, the computer 408 graphically displays on the display device 304 an indication of the C-arm 1308 position relative to the optimal position, and lets the user move and orient the C-arm 1308. As the C-arm 1308 is moved by the user, the graphical display on the display device 304 is continuously updated to show the user how close the C-arm 1308 is to its optimal position. The location of the C-arm 1308 can be tracked by the optical markers 2202,2204 on the calibration ring 2200 or some other trackable markers that are positioned on the C-arm. One example of the graphical display is illustrated in FIG. 25.

The left image displays an x-y-z coordinate of the C-arm 1308. The dotted circle represents the optimal position of the C-arm 1308. The center of the dotted circle represents the optimal X-Y position with the size of the dotted circle representing the optimal Z position. The solid circle represents the actual position of the C-arm 1308. As the user moves the C-arm 1308, the solid circle moves and changes its size to indicate its actual 3D (X-Y-Z) position relative to the optimal position.

The right image displays a Yaw-Pitch-Roll coordinate of the C-arm 1308. The dotted circle represents the optimal orientation of the C-arm 1308. The center of the dotted circle represents the optimal Yaw-Pitch position with the size of the dotted circle representing the optimal Roll position. The solid circle represents the actual position of the C-arm 1308 in terms of Yaw, Pitch and Roll. As the user moves the C-arm 1308, the solid circle moves and changes its size to indicate its 3D orientation relative to the optimal position.

Once the solid circle on both coordinates have been aligned with the respective dotted circles, the imaging device 1304 is ready to take the appropriate image. For example, if L1-AP 2402 had been selected by the user, the imaging device 2300 takes the AP image. It can be done by actuating an appropriate button on the imaging device or instructions from the computer 408 can be sent to do so.

Alternatively, once a vertebral level is selected, the computer 408 can send instructions to the imaging device 2300 to take both the optimal AP and lateral images based on the stored optimal position and orientation that have been determined through steps 2102-2110.

In one embodiment, for every image taken and stored by the imaging device 2300, the computer 408 also stores in the memory 410 the image as well as the position and orientation information of the C-arm 2316. That can be achieved either through the optical markers 2310 and 2202-2204, or through the imaging device's internal positioning elements such as encoders in the motors controlling every axis and 3D position of the C-arm 2316.

As the images are being taken, additional vertebral levels may become available in the newly acquired images. For example, as optimal L1 images are being taken, those images may contain new levels such as L4. In one aspect of the present invention, the computer 408 stores the newly acquired images (both AP and lateral) and their position and orientation in the memory 410 and then repeats steps 2102 through 2112 for the new vertebral level if the new level was identified in step 2104 as of interest.

In another aspect of the present invention, the computer 408 may refine the optimal 3D position and orientation data which have already been obtained. In the same example, the computer 408 may repeat steps 2102 through 2112 for L2 and L3 based on the newly acquired AP and lateral images. Since the images were taken based on the optimal 3D position and orientation data for L1, they may also contain a more optimally aligned levels for L2 and L3. Thus, the refined 3D and orientation positions for L2 and L3 will likely be even more accurate than before.

In step 2112, in addition to input buttons 2402,2404 for the old levels (e.g., L1-L3), the computer 408 displays the graphical representation of input buttons 2410,2412 for the new level (e.g., AP and lateral for L4) on the display device 304.

As can be appreciated, the method described above substantially reduces the setup time for positioning an x-ray imaging device in the operating room as only two fluoro shots (one set of AP and lateral images) are needed for each vertebral level, instead of requiring 10 or more. This advantageous feature yields many benefits including a substantial reduction in procedure time, substantial reduction in radiation exposure for the patient as well as the medical professionals, and reduced cost for the procedure due to less time being required for the procedures. Perhaps more importantly, because the present invention allows more optimal images to be taken, it allows the physician to place the implants more accurately, which leads to better patient outcome in many surgeries.

Although several embodiments of the invention have been disclosed in the foregoing specification, it is understood that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. For example, while the invention has been described with reference to lumbar spine, it can applicable to any body implant (with virtual 3-D model of the implant), any body structure or other other areas of the spine including cervical spine and thoracic spine as well as any other body part that requires A-P and lateral images such as knees It is further envisioned that features from one embodiment may be combined or used with the features from a different embodiment described herein. Moreover, although specific terms are employed herein, as well as in the claims which follow, they are used only in a generic and descriptive sense, and not for the purposes of limiting the described invention, nor the claims which follow. The entire disclosure of each patent and publication cited herein is incorporated by reference, as if each such patent or publication were individually incorporated by reference herein. Various features and advantages of the invention are set forth in the following claims.

What is claimed is:

1. An imaging system for taking optimal 2D x-ray images of a vertebral body of a patient, the system comprising:
a movable imaging arm configured to take 2D x-ray images at a plurality of orientations relative to the vertebral body;
a processor coupled to the movable imaging arm;
a memory coupled to the processor;
an image control module stored in the memory and executable by the processor;
wherein under the control of the processor, the image control module is configured to:
receive, from the memory, test images including:
a first x-ray image of the vertebral body taken by the movable imaging arm;
a second x-ray image of the vertebral body taken by the movable imaging arm at a different angle than the first x-ray image; and
3D positions of the movable imaging arm at the time of taking the first and second x-ray images;
segment, among a plurality of vertebral bodies contained in the x-ray test images, vertebral bodies;
retrieve a 3D model of the vertebral body from the memory;
align the retrieved 3D model against the vertebral body contained in the test images by performing a fluoro-3D merge using the 3D positions of the movable imaging arm for the first and second x-ray images;
automatically determine a 3D position of the movable imaging arm for purposes of taking future optimal A-P and lateral x-ray images based on the aligned 3-dimensional model.

2. The imaging system of claim 1, wherein the image control module is configured to receive from a user an identification of a level of a single vertebral body among the plurality of vertebral bodies contained in the x-ray test images and the image control module automatically identifies the remaining vertebral bodies in the x-ray test images.

3. The imaging system of claim 2, wherein the image control module is configured to repeat the 3D position determination of the movable imaging arm for a next level of the vertebral bodies to be imaged.

4. The imaging system of claim 1, wherein the image control module is configured to scale the size of the 3D model of the vertebral body to correspond to the vertebral body contained in the test images.

5. The imaging system of claim 1, wherein the image control module is configured to align the retrieved 3D model against the vertebral body by performing a fluoro-CT merge using a CT image of the vertebral body.

6. The imaging system of claim 1, wherein the image control module is further configured to:
receive the optimal A-P and lateral x-ray images based on the determined 3D position and orientation of the imaging arm;
identify an additional vertebral body contained in the received optimal A-P and lateral x-ray images;
retrieve a 3D model of the identified additional vertebral body from the memory;
3-dimensionally align the retrieved 3D model against the received optimal A-P and lateral x-ray images;
determine the 3D position and orientation of the imaging arm for taking optimal A-P and lateral x-ray images for the identified additional vertebral body based on the aligned 3D model of the additional vertebral body.

7. The imaging system of claim 1, wherein, prior to receiving test images, the image control module is configured to derive the 3D model from a 3D scan of the patient.

8. The imaging system of claim 1, further comprising a display device, wherein the image control module is further configured to:
track the 3D position of the imaging arm through navigation markers; and
display in the display device a graphical representation of the extent of the imaging arm alignment relative to the optimal 3D position.

9. The imaging system of claim 8, wherein the image control module is configured to:
display a graphical representation of an optimal 3D position of the imaging arm on a coordinate system;
display a graphical representation of a current position and orientation of the imaging arm on the same coordinate system;
continuously update the location of the graphical representation of the current position of the imaging arm as the imaging arm is being moved.

10. The imaging system of claim 8, wherein the image control module is configured to continuously update by:
displaying a circle in the center of a coordinate system as the graphical representation of the optimal position of the imaging arm;
varying the size of the graphical representation of the current position of the imaging arm as the imaging arm moves in either X, Y or Z direction.

11. The imaging system of claim 8, wherein the image control module is configured to continuously display the graphical representation by displaying a graphical representation of how closely the imaging arm is aligned with the optimal 3D position of the imaging arm relative to the identified vertebral body to be imaged.

12. The imaging system of claim 1, wherein the image control module is configured to:
continuously track the position of the imaging arm through the navigation markers; and
automatically send instructions to the imaging device based on the determined 3D position of the imaging arm,
wherein the imaging arm is registered to the tracking subsystem without the use of any radio-opaque markers.

13. An imaging system for taking optimal 2D x-ray images of a vertebral body of a patient, the system comprising:
a tracking subsystem having a plurality of cameras;
a movable imaging arm having a plurality of optical navigation markers and configured to take 2D x-ray images at a plurality of orientations relative to the vertebral body;
a processor coupled to the movable imaging arm and the tracking subsystem;
a memory coupled to the processor;
an image control module stored in the memory and executable by the processor;
wherein under the control of the processor, the image control module is configured to:
register the movable imaging arm to the tracking subsystem by detecting navigation markers located on the imaging arm;
receive, from the memory, test images including:
a first x-ray image of the vertebral body taken by the movable imaging arm;
a second x-ray image of the vertebral body taken by the movable imaging arm at a different angle than the first x-ray image; and
3D positions of the movable imaging arm at the time of taking the first and second x-ray images;
segment, among a plurality of vertebral bodies contained in the x-ray test images, vertebral bodies;
retrieve a 3D model of the vertebral body from the memory;
align the retrieved 3D model against the vertebral body contained in the test images by performing a fluoro-3D merge using the 3D positions of the movable imaging arm for the first and second x-ray images;
automatically determine a 3D position of the movable imaging arm for purposes of taking future optimal A-P and lateral x-ray images based on the aligned 3-dimensional model.

14. The imaging system of claim 13, wherein the image control module is configured to receive from a user an identification of a level of a single vertebral body among the plurality of vertebral bodies contained in the x-ray test images and the image control module automatically identifies the remaining vertebral bodies in the x-ray test images.

15. The imaging system of claim 13, wherein the image control module is configured to scale the size of the 3D model of the vertebral body to correspond to the vertebral body contained in the test images.

16. The imaging system of claim 13, wherein the image control module is configured to align the retrieved 3D model against the vertebral body by performing a fluoro-CT merge using a CT image of the vertebral body.

17. The imaging system of claim 13, wherein, prior to receiving test images, the image control module is configured to derive the 3D model from a 3D scan of the patient.

18. The imaging system of claim 13, further comprising a display device, wherein the image control module is further configured to:
track the 3D position of the imaging arm through navigation markers; and
display in the display device a graphical representation of the extent of the imaging arm alignment relative to the optimal 3D position.

19. The imaging system of claim 18, wherein the image control module is configured to:
display a graphical representation of an optimal 3D position of the imaging arm on a coordinate system;
display a graphical representation of a current position and orientation of the imaging arm on the same coordinate system;
continuously update the location of the graphical representation of the current position of the imaging arm as the imaging arm is being moved.

20. The imaging system of claim 18, wherein the image control module is configured to continuously display the graphical representation by displaying a graphical representation of how closely the imaging arm is aligned with the optimal 3D position of the imaging arm relative to the identified vertebral body to be imaged.
